SEQUENCE LISTING (18 sequences)

SEQ ID NO: 1 - protein, 5 aa, Homo sapiens
SYGMH

SEQ ID NO: 2 - protein, 17 aa, Homo sapiens
VIWFDGTKKYYTDSVKG

SEQ ID NO: 3 - protein, 15 aa, Homo sapiens
DRGIGARRGPYYMDV

SEQ ID NO: 4 - protein, 11 aa, Homo sapiens
RASQSISSYLN

SEQ ID NO: 5 - protein, 7 aa, Homo sapiens
AASSLQS

SEQ ID NO: 6 - protein, 9 aa, Homo sapiens
QQSYSTPLT

SEQ ID NO: 7 - protein, 30 aa, Homo sapiens
QVQLVESGGGVVQPGRSLRLSCAASGFAFS

SEQ ID NO: 8 - protein, 14 aa, Homo sapiens
WVRQAPGKGLEWVA

SEQ ID NO: 9 - protein, 32 aa, Homo sapiens
RFTISRDNSKNTLYLQMNTLRAEDTAVYYCAR

SEQ ID NO: 10 - protein, 11 aa, Homo sapiens
WGKGTTVTVSS

SEQ ID NO: 11 - protein, 23 aa, Homo sapiens
DIQMTQSPSSLSASVGDRVTITC

SEQ ID NO: 12 - protein, 15 aa, Homo sapiens
WYQQKPGKAPKLLIY

SEQ ID NO: 13 - protein, 32 aa, Homo sapiens
GVPSRFSGSGSGTDFTLTISSLQPEDFATYYC

SEQ ID NO: 14 - protein, 11 aa, Homo sapiens
FGGGTKVEIKR

SEQ ID NO: 15 - protein, 124 aa, Homo sapiens
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 16 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 17 - DNA, 372 bp, Homo sapiens
caggtgcagctggtggagtccggaggaggagtggtgcagccaggccggtccctgagactgtcttgtgccgccagcggcttcgcctttagctcctatggcatgcactgggtgaggcaggcccccggcaagggcctggagtgggtggccgtgatctggttcgacggcaccaagaagtactatacagatagcgtgaagggcaggtttaccatctcccgcgacaactctaagaatacactgtacctccagatgaataccctgcgcgccgaggacacagccgtgtactattgcgccagggatcgcggaatcggcgccagaagaggaccttactatatggacgtgtggggcaagggcaccacagtgaccgtgtctagc

SEQ ID NO: 18 - DNA, 321 bp, Homo sapiens
gacatccagatgacccagagcccaagctccctgtctgcaagtgtgggcgatcgggtcaccatcacatgtagagcctcacagagcatttctagttacctgaactggtatcagcagaagccagggaaagctcccaagctgctgatctacgccgcttcaagcctgcagtctggagtgcccagtcgattctccggctctgggagtggaacagactttactctgaccatttcctctctgcagcctgaggatttcgccacttactattgccagcagtcatatagcaccccactgacatttggcgggggaacaaaagtggagatcaag

(19) United States
(12) Patent Application Publication
(10) Pub. No.: US 2023/0030950 A1
(43) Pub. Date: Feb. 2, 2023

(54) TREATMENT OF NEURODEGENERATIVE DISEASES USING ULTRASOUND AND AMYLOID-BETA ANTIBODIES

(71) Applicant: THE UNIVERSITY OF QUEENSLAND, Queensland (AU)

(72) Inventors: Juergen GOETZ, Brisbane (AU); Gerhard LEINENGA, Brisbane (AU)

(73) Assignee: THE UNIVERSITY OF QUEENSLAND, Queensland (AU)

(21) Appl. No.: 17/782,159

(22) PCT Filed: Dec. 3, 2020
(86) PCT No.: PCT/AU2020/051320

§ 371 Date: Jun. 2, 2022

(30) Foreign Application Priority Data
Dec. 3, 2019 (AU) 2019904578

(51) Int. Cl. A61M 37/00; C07K 16/18; A61K 41/00; A61P 25/28

(52) U.S. Cl. CPC A61M 37/0092; C07K 16/18; A61K 41/0023; A61P 25/28; A61M 2210/0693; A61M 2202/07; C07K 2317/565; C07K 2317/34; C07K 2317/92; C07K 2317/76; A61K 2039/505

(57) ABSTRACT

The present invention relates to antigen binding sites, compositions and uses thereof in the treatment of conditions associated with pathogenic proteins. In one example the condition is Alzheimer's disease.

### FIELD OF THE INVENTION

The invention relates to methods for treating or preventing neurodegenerative diseases, particularly those associated with amyloid-beta.

### CROSS REFERENCE TO EARLIER APPLICATION

This application claims priority from Australian provisional application no. 2019904578, the entire contents of which are hereby incorporated by reference in their entirety.

### BACKGROUND OF THE INVENTION

The deposition of amyloid-β in the brain is thought to be a key and initiating step in the development of Alzheimer's disease (AD) so development of AD therapies have focused on approaches that might clear amyloid from the brain. Therapies based on monoclonal antibodies are currently in clinical trials for AD to enhance amyloid-β (Aβ) clearance from brain.

Results of clinical trials in AD patients have been unimpressive. This may reflect the poor penetration of therapeutic to brain, resulting in inadequate target engagement. Peripherally injected antibodies have low penetration of the blood-brain barrier (BBB), with only 0.1%-0.2% entering brain. Because of the poor delivery of antibodies to the brain therapeutic antibodies are often given at doses 1,000-fold greater than the concentration needed to achieve adequate binding of the target if the BBB was not present and these high doses in clinical trials increase the prevalence of adverse events such as amyloid-related imaging abnormalities (ARIA) which can occur in up to 100 percent of patients treated with high doses of antibody.

Recently, in a phase one clinical trial a striking reduction in amyloid was reported following a year of monthly intravenous infusions of aducanumab in mild AD patients at doses ranging from 3-10 mg/kg. Aducanumab is one of several antibodies against amyloid-beta for which clinical trial data exists. While the reduction in amyloid in the aducanumab trial was impressive, results obtained with other antibodies have failed to reduce amyloid significantly and all trials have seen adverse events occur. The occurrence of adverse events and high cost of antibodies might prevent antibodies being applied in primary prevention trials. One obstacle in treating AD using this approach is presented by the blood-brain barrier (BBB) which limits the entry of antibodies increasing cost and potentially reducing the efficacy of passive immunotherapy against Aβ.

There is a need for new and/or improved methods for the treatment or prevention of neurodegenerative diseases, preferably those associated with amyloid-beta.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the invention provides for a method of delivering an antigen binding site that binds to or specifically binds to an amyloid beta (β) protein in a subject comprising:

administering to the subject an antigen binding site that binds to or specifically binds to an amyloid beta protein, and administering acoustic energy to a region of the brain of the subject;

wherein the application of acoustic energy acts as a means to permit or facilitate the antigen binding site to pass through the blood-brain barrier (BBB) of the subject, thereby delivering the antigen binding that binds to or specifically binds to an amyloid beta protein.

Preferably, the amyloid beta protein may be in any form, for example, soluble Aβ, Aβ oligomer or present in amyloid plaques. In one embodiment, the amyloid beta protein is in the form of an aggregate, preferably including insoluble fibrils and soluble oligomers.

As used herein a pathogenic form of an amyloid beta protein may be a fibril or a plaque. Preferably, the pathogenic form is a plaque.

The amyloid beta protein may be select from amyloid beta, amyloid fragments, amyloid precursor protein, and amyloid precursor protein fragments.

In any aspect of the present invention, the application of acoustic energy acts as a means to permit or facilitate the antigen binding site to pass through the blood-brain barrier (BBB) of the subject

In any aspect of the present invention, the acoustic energy is ultrasound. The ultrasound may be scanning ultrasound (SUS) or non-scanning ultrasound. In an embodiment, the SUS or non-scanning ultrasound is administered with microbubbles to disrupt the blood-brain barrier. The administration of microbubbles may be before, after or during the administration of SUS or non-scanning ultrasound. In some instances, the antibody or antigen-binding fragment is administered to the subject before, at the same time and/or after the subject has received scanning ultrasound (SUS) or non-scanning ultrasound.

In another aspect the present invention provides a method of improving cognitive function in a subject, the method comprising, consisting essentially of or consisting of the steps of:

administering to the subject an antigen binding site that binds to or specifically binds to an amyloid beta protein;

identifying a region of the brain of the subject to which acoustic energy is to be applied; and

applying a clinically safe level of acoustic energy to the region, thereby saturating or substantially saturating the region with acoustic energy;

thereby improving cognitive function in the subject.

In any aspect of the present invention, the subject may have impaired cognitive function. Impaired cognitive function may be determined by any method as described herein. Further, in any method or use of the invention, the subject may be identified as having impaired cognitive function. In any method of the invention, the method further comprises a step of identifying an individual with impaired cognitive function.

In another aspect the present invention provides a method of improving cognitive function in a subject with a condition associated with a pathological form of an amyloid beta protein, the method comprising, consisting essentially of or consisting of the steps of:

administering to a subject an antigen binding site that binds to or specifically binds to an amyloid beta protein;

identifying a region of the brain of the subject to which acoustic energy is to be applied; and

applying a clinically safe level of acoustic energy to the region, thereby saturating or substantially saturating the region with acoustic energy;

thereby improving cognitive function in the subject.

In any aspect of the invention, the condition or disease for treatment is one associated with or caused by a pathological form of an amyloid beta protein. Preferably, the amyloid beta protein is in the form of an oligomer, aggregate or deposit. The condition or disease is any one described herein.

In another aspect the present invention provides a method of improving memory, motor skills and/or executive functions in a subject with impaired memory function, the method including the steps of:

administering to a subject an antigen binding site that binds to or specifically binds to an amyloid beta protein;

identifying a region of the brain of the subject to which acoustic energy is to be applied; and

applying a clinically safe level of acoustic energy to the region, thereby saturating or substantially saturating the region with acoustic energy;

thereby improving memory, motor skills and/or executive functions in the subject.

In another aspect, the present invention provides a method of improving memory, motor skills, executive functions and/or cognitive function in a subject with impaired memory and/or cognitive function, the method including the steps of:

providing a subject with impaired memory, motor skills, executive functions, and/or cognitive function;

administering to the subject an antigen binding site that binds to or specifically binds to an amyloid beta protein;

identifying a region of the brain of the subject to which acoustic energy is to be applied; and

applying a clinically safe level of acoustic energy to the region, thereby saturating or substantially saturating the region with acoustic energy;

thereby improving memory, motor skills, executive functions and/or cognitive function in the subject.

Preferably, identifying a region of the brain as described herein includes determining a volume of the brain on the basis of symptoms displayed by the subject, typically clinically observable or biochemically detectable symptoms, or determining a volume of the brain on the basis of a known association with an amyloid beta protein, preferably in pathogenic form, in particular those associated with protein oligomers, aggregates or deposits, or determining a volume of the brain including a volume surrounding an site having an amyloid beta protein in a pathogenic form, such as oligomers, an aggregate or deposit.

In any aspect, the method of the invention further includes determining a plurality of discrete application sites for application of acoustic energy to saturate or substantially saturate the region with acoustic energy.

In any aspect, the method further includes determining a scanning path along which acoustic energy is to be applied to saturate or substantially saturate the region with acoustic energy. Preferably, the method further includes determining a plurality of discrete application sites for application of acoustic energy along the scanning path.

Typically, applying a clinically safe level of acoustic energy to the region includes providing acoustic energy continuously, or at application sites, along a scanning path.

In one embodiment, the scanning path is defined by a pre-determined pattern. The scanning path may be selected from the group consisting of linear, serpentine, a raster pattern, spiral and random.

Each application site may be spaced along the scanning path or each subsequent application site may overlap with the previous application site.

Applying a clinically safe level of acoustic energy to the region, includes applying acoustic energy at an application site such that a corresponding treatment volume is therapeutically affected by acoustic energy, and wherein saturating or substantially saturating the region with acoustic energy includes applying acoustic energy at a plurality of discrete application sites or one or more extended application sites such that the corresponding treatment volume(s) correspond substantially with the region.

The plurality of application sites may be selected such that treatment volumes of at least some sites overlap to form a group of treatment volumes that corresponds substantially with the region.

The plurality of application sites may be selected such that their corresponding treatment volumes overlap to form a contiguous treatment volume that corresponds substantially with the region.

A region of the brain may the entire brain, hemisphere, forebrain or a region of the brain of the subject known to be associated with a condition involving the presence of proteins adopting pathogenic structures in an extracellular region. Such structures may be oligomers, aggregates and/or deposits. The region may be any one or more of the following cerebrum, cerebral hemisphere, telencephalon, forebrain, cortex, frontal lobe, prefrontal cortex, precentral gyrus, primary motor cortex, premotor cortex, temporal lobe, auditory cortex, inferior temporal cortex, superior temporal gyrus, fusiform gyrus, parahippocampal gyrus, entorhinal cortex, parietal lobe, somatosensory cortex, postcentral gyrus, occipital lobe, visual cortex, insular cortex, cingulate cortex, subcortical, hippocampus, dentate gyrus, cornu ammonis, amygdala, basal ganglia, striatum, caudate, putamen, nucleus accumbens, olfactory tubercle, globus pallidus, subthalamic nuclei, piriform cortex, olfactory bulb, fornix, mammillary bodies, basal forebrain, nucleus basalis Meynert, diencephalon, thalamus, hypothalamus, midbrain, tectum, tegmentum, substantia nigra, hindbrain, myelencephalon, medulla oblongata, metencephalon, pons, cerebellum, spinal cord, brain stem and cranial nerves.

In a subject identified as having Alzheimer's disease the region may be selected from the group consisting of cerebrum, cerebral hemisphere, telencephalon, forebrain, cortex, frontal lobe, prefrontal cortex, precentral gyrus, temporal lobe, auditory cortex, inferior temporal cortex, superior temporal gyrus, fusiform gyrus, parahippocampal gyrus, entorhinal cortex, insular cortex, cingulate cortex, subcortical, hippocampus, dentate gyrus, cornu ammonis, amygdala, piriform cortex, olfactory bulb, fornix, mammillary bodies, basal forebrain and nucleus basalis of Meynert.

In any embodiment of the invention, the region is not solely identified as a plaque. The region may be an aggregate or deposit of pathological protein.

As used herein the acoustic energy may provide conditions for an increase in the permeability of the blood-brain barrier, or activating microglia. Conditions for an increase in the permeability of the blood-brain barrier are described further herein.

Preferably, a clinically safe level of acoustic energy does not result in detectable heating, brain swelling, red blood cell extravasation, haemorrhage or edema.

A subject with impaired cognitive function, motor skills, executive functions and/or memory function may be identified as having a neurodegenerative disease associated with, or caused by, the presence, over-expression or accumulation of an amyloid beta protein. As used herein, pathological amyloid beta protein refers to a form or an amount of amyloid beta protein that is not present in a normal individual, i.e. one without impaired cognitive function, motor skills, executive functions and/or memory function.

In any embodiment, the subject in need thereof has mainly, or only, detectable cortical plaques. The subject may or may not have detectable plaques in the hippocampus.

Typically, an improvement in cognitive function or memory is determined by standardised neuropsychological testing.

In any aspect of the present invention, the administration of the antigen binding site and the application of acoustic energy may be sequential or concurrent. Alternatively, administration and application may be done at different times. In an embodiment, the SUS is administered to the entire or whole brain as a means to allow for an antigen binding site to pass through the blood-brain barrier.

In any aspect of the present invention, the antigen binding site with SUS inhibits or prevents the accumulation, or deposition of amyloid beta protein in the central nervous system. Preferably, the administration of the antigen binding site with or without SUS improves cognitive function in a subject with a condition associated with, or caused by, an amyloid beta protein.

In any aspect, an antigen binding site that binds to amyloid-β has an affinity that is not statistically different to the antibody BIIB037 (aducanumab). Typically, the antigen binding site of the invention that binds to amyloid-β binds to the same epitope on amyloid-β as BIIB037 (aducanumab). Preferably, an antigen binding site of the invention competes with BIIB037 (aducanumab) for binding to amyloid-β. In any aspect, the antigen binding site is aducanumab.

In any aspect, an antigen binding site binds aggregated forms of Aβ, and may also preferentially bind parenchymal amyloid over vascular amyloid.

In any aspect, an antigen binding site binds a linear epitope formed by amino acids 3-7 of the Aβ peptide, preferably the liner epitope comprises, consists essentially of or consists of the amino acid sequence EFRHD.

In any aspect, an antigen binding site binds to Aβ fibrils, with an EC50 of >1 μM for monomeric Aβ1-40 and/or an EC50 of about 0.2 nM, 0.2 nM or about 0.1 nM for fibrillar Aβ1-42 (or any other affinity value described herein).

In any aspect, the antigen binding site comprises an antigen binding domain of an antibody, wherein the antigen binding domain binds to or specifically binds to amyloid-beta, wherein the antigen binding domain comprises:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(ii) a VH comprising a sequence at least about 95% or 96% or 97% or 98% or 99% identical to a sequence set forth in SEQ ID NO: 15;
(iii) a VH comprising a CDR1 comprising a sequence set forth in SEQ ID NO: 1, a CDR2 comprising a sequence set forth between in SEQ ID NO: 2 and a CDR3 comprising a sequence set forth in SEQ ID NO: 3; or
(iv) a VH comprising a sequence set forth in SEQ ID NO: 15;
and,
(v) a VL comprising a CDR1 comprising a sequence at least about 80%, at least 85%, at least 90%, at least 92%, at least 95%, at least 97%, at least 99% identical to a sequence set forth in SEQ ID NO: 4, a CDR2 comprising a sequence at least about 80%, at least 85%, at least 90%, at least 92%, at least 95%, at least 97%, at least 99% identical to a sequence set forth in SEQ ID NO: 5 and a CDR3 comprising a sequence at least about 80%, at least 85%, at least 90%, at least 92%, at least 95%, at least 97%, at least 99% identical to a sequence set forth in SEQ ID NO: 6;
(vi) a VL comprising a sequence at least about 95% identical to a sequence set forth in SEQ ID NO: 16;
(vii) a VL comprising a CDR1 comprising a sequence set SEQ ID NO: 4, a CDR2 comprising a sequence set forth in SEQ ID NO: 5 and a CDR3 comprising a sequence set forth in SEQ ID NO: 6; or
(viii) a VL comprising a sequence set forth in SEQ ID NO: 16.

In any aspect, the antigen binding site comprises an antigen binding domain of an antibody, wherein the antigen binding domain binds to or specifically binds to amyloid-beta, wherein the antigen binding domain comprises:
(i) a VH comprising a CDR1 comprising a sequence set forth in SEQ ID NO: 1 with 0 to 5 amino acid insertions, deletions, substitutions or additions (or a combination thereof), a CDR2 comprising a sequence set forth between in SEQ ID NO: 2 with 0 to 5 amino acid insertions, deletions, substitutions or additions (or a combination thereof), and a CDR3 comprising a sequence set forth in SEQ ID NO: 3 with 0 to 5 amino acid insertions, deletions, substitutions or additions (or a combination thereof); or
(ii) a VH comprising a sequence set forth in SEQ ID NO: 15 with 0 to 5 amino acid insertions, deletions, substitutions or additions (or a combination thereof);
and,
(iii) a VL comprising a CDR1 comprising a sequence set SEQ ID NO: 4 with 0 to 5 amino acid insertions, deletions, substitutions or additions (or a combination thereof), a CDR2 comprising a sequence set forth in SEQ ID NO: 5 with 0 to 5 amino acid insertions, deletions, substitutions or additions (or a combination thereof), and a CDR3 comprising a sequence set forth in SEQ ID NO: 6 with 0 to 5 amino acid insertions, deletions, substitutions or additions (or a combination thereof); or
(iv) a VL comprising a sequence set forth in SEQ ID NO: 16 with 0 to 5 amino acid insertions, deletions, substitutions or additions (or a combination thereof).

In some embodiments of the above aspect, the relevant amino acid sequence may have from 0 to 4, preferably from 0 to 3, preferably from 0 to 2, preferably from 0 to 1 amino acid insertions, deletions, substitutions or additions (or a combination thereof).

As described herein, the antigen binding site may be in the form of:

a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3; or
(iv) one of (i) or (ii) linked to a protein that binds to amyloid beta.

Further, as described herein, the antigen binding site may be in the form of:
(i) a diabody;
(ii) a triabody;
(iii) a tetrabody;
(iv) a Fab;
(v) a F(ab′)2;
(vi) a Fv;
(vii) one of (i) to (vi) linked to a constant region of an antibody, Fc or a heavy chain constant domain (CH) 2 and/or CH3; or
(viii) one of (i) to (vi) linked to a protein that binds to amyloid beta.

Further, as described herein, the antigen binding site may be in the form of:
(i) IgG1;
(ii) IgG2a, IgG2b, IgG3;
(iii) one of (i) to (ii) linked to a constant region of an antibody, Fc or a heavy chain constant domain (CH) 2 and/or CH3;
(iv) one of (i) to (vi) linked to a protein that binds to amyloid-beta.

The foregoing antigen binding sites can also be referred to as antigen binding domains of antibodies.

Preferably, an antigen binding site as described herein is an antibody or antigen binding fragment thereof. Typically, the antigen binding site is an antibody, for example, a monoclonal antibody.

In any embodiment of the invention, the antigen binding site may be a synthetic binding site. For example, the binding site may be chimeric, humanized, human, synhumanized, primatized, de-immunized or a composite antigen binding site.

In any aspect of the present invention, the antigen binding site is any antigen binding site as described herein.

As used herein, the complementarity determining region sequences (CDRs) of an antigen binding site of the invention are defined according to the IMGT or the Chothia numbering system.

Reference herein to a protein or antibody that “binds to” an amyloid beta protein provides literal support for a protein or antibody that “binds specifically to” or “specifically binds to” an amyloid beta protein.

The present invention also provides antigen binding domains or antigen binding fragments of the foregoing antibodies.

An antigen binding site of the present invention as described herein may be used in any method, use or composition of the invention as described herein.

An antigen binding site as described herein may be purified, substantially purified, isolated and/or recombinant.

An antigen binding site of the invention may be part of a supernatant taken from media in which a hybridoma expressing an antigen binding site of the invention has been grown.

In another aspect, the invention provides a method for treating, delaying, reducing, inhibiting or preventing the accumulation or deposition of pathological protein aggregates in the central nervous system in a subject, comprising

administering an antigen binding site as described herein; and

administering acoustic energy to the brain of a subject,

wherein the application of acoustic energy acts as a means to permit or facilitate the antigen binding site to pass through the blood-brain barrier (BBB),

treating, delaying, reducing, inhibiting or preventing the accumulation or deposition of pathological protein aggregates in the central nervous system in a subject.

In another aspect, the invention provides a use of an antigen binding site as described herein, in the preparation of a medicament for treating, inhibiting, delaying or reducing the progression of a disease or condition associated with amyloid beta protein in a subject in need thereof in combination with applying acoustic energy to the subject in need thereof, preferably scanning ultrasound (SUS).

In another aspect, the invention provides a use of an antigen binding site as described herein, in the preparation of a medicament for treating, inhibiting, delaying or reducing the progression of a disease or condition associated with amyloid beta protein in a subject in need thereof who has received or who is receiving an application of acoustic energy, preferably scanning ultrasound (SUS).

In another aspect, the invention provides an antigen binding site as described herein, for use in treating, inhibiting, delaying or reducing the progression of a disease or condition associated with amyloid beta protein in a subject in need thereof who has received or who is receiving an application of acoustic energy, preferably scanning ultrasound (SUS).

Typically, a method of the invention also includes the step of administering an agent to promote the increase in permeability of the blood-brain barrier. In a preferred form that agent promotes cavitation. Typically, a use or antigen binding site for use as described herein applies to a subject who has received or who is receiving an agent to promote the increase in permeability of the blood-brain barrier. An agent that promotes cavitation may be a microbubble agent as described herein.

In any embodiment of the invention, the method may further comprise a step of administering a microbubble agent to the subject. Administration of microbubbles may be before, after or during the administration of SUS.

The microbubble may be provided to the subject by continuous infusion or a single bolus. The infusion may occur sequentially to, or following the start of, or simultaneously with, the application of the ultrasound.

In an embodiment, the step of applying the acoustic energy is repeated.

Any method of the invention described herein may also further include the step of determining that the permeability of the blood-brain barrier has increased.

The acoustic energy may be applied in a method of the invention at a pressure greater than 0.4 MPa. Typically this pressure is used when application of the acoustic energy is outside the skull, i.e. transcranially. Otherwise, the acoustic energy may be applied with a mechanical index of between 0.1 and 2.

In a further aspect, the present invention provides an apparatus configured to perform any one or more of the methods described herein. The apparatus may comprise any one or more of the following: an antibody delivery device configured to deliver an antibody to a subject (preferably any antibody as described herein), an acoustic energy emitter configured to emit acoustic energy for delivery to a region of the brain of the subject, a microbubble delivery device configured to deliver microbubbles to a region of the brain of the subject for disrupting the blood-brain barrier, and a controller that may control any one or more of the antibody delivery device, the acoustic energy emitter, and the microbubble delivery device. The apparatus may be used in conjunction with an imaging device, such as an MRI device, a positron emission tomography (PET) device, a computerized tomography (CT) or computerized axial tomography (CAT) device, or an ultrasound device. The apparatus may also be used in conjunction with an imaging contrast agent delivery device configured to deliver an imaging contrast agent to a region of the brain of the subject to aid in imaging of the brain by the imaging device. The imaging device and the imaging contrast agent may be controlled by the controller.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Study overview and results of APA test and retest. (A) Overview of the study with timeline. (B) Active place avoidance test (APA) in which mice must use spatial cues to avoid a shock zone indicated as a red triangle. (C) APP23 mice had impaired performance in the APA test in terms of number of shocks received. (D) APP23 mice had shorter time to first entry to the shock zone as determined by two-way ANOVA. (E) APP23 mice did not show significant impairment in the measure number of entries (E) or maximum time of avoidance (F), but were impaired on the measures time to second entry (G) and proportion of time spent in the opposite quadrant to the shock zone (H). APP23 mice were then assigned to treatment groups based on matched performance on day 5 of the APA (I). APA retest was performed after four once-per-week treatments with changes to room cues, shock zone location, and the direction of rotation (J). Effect of treatment on number of entries (M), maximum time of avoidance (N), time to second entry (0) and proportion of time spent in the opposite quadrant to the shock zone (P). Data is represented as mean±SEM. Statistical differences: *p<0.05, **p<0.01, ***p<0.001, ****p<0.0001, $=simple effect comparing wild-type vs sham p<0.05, #=simple effect comparing SUS vs sham p<0.05, &=simple effect comparing SUS+Adu vs sham p<0.05. Sham N=10, Adu N=11, SUS N=11, SUS+Adu N=10, WT N=12. Data were analyzed with a two-way ANOVA and follow-up Holm-Sidak tests for simple effects.

FIG. 2. Treatment strategies reduce plaques in APP23 mice. (A) Representative Campbell-Switzer silver staining for amyloid plaques in the four treatment groups. Plaques stained black are more diffuse, while amber plaques are compact and discrete. The black box shows the entire hemisphere (scale bars: 1 mm). Insert outlined in green shows higher magnification view of dorsal hippocampus (scale bars: 500 μm), and the red inset shows higher magnification image of temporal cortex (scale bars: 100 μm). (B) There was a significant reduction of plaques in the cortex of SUS+Adu-treated mice, driven largely by reduction in black plaques (C) as area, number and size of amber plaque was less affected by treatment (D, E,I; in E and I cortex results are shown in the left column of each pair and hippocampus is shown in the right column of each pair). Plaque load in the hippocampus was reduced by Adu, SUS and SUS+Adu (F) and hippocampal black plaques (G) and amber plaques (H) were analyzed separately. A significant correlation was found between amyloid levels measured by histology and by ELISA (J-M). Data is represented as mean±SEM. Statistical differences: *p<0.05, **p<0.01, ***p<0.001. Data were analyzed with a one-way ANOVA and follow-up Holm-Sidak tests. Sham N=10, Adu N=9, SUS N=8, SUS+Adu N=9.

FIG. 3. Aducanumab analog does not affect levels of cerebral amyloid angiopathy (CAA) in APP23 mice. (A) Representative Campbell-Switzer silver staining shows CAA in the cortex identified by a rod-like appearance, as well as meningeal CAA identified as open circles on top of the cortex. (B) Adu, whether administered with or without SUS, had no effect on the CAA number, average size, or percent area occupied by CAA. Data is represented as mean±SEM. Statistical differences: *p<0.05. Data were analyzed with a one-way ANOVA and t-test.

FIG. 4. Scanning ultrasound (SUS) increases levels of the Aducanumab analog in the brain. (A) Fluorescently labeled Aducanumab analog is detectable in the brain when viewed in the entire brain (scale bars 1 mm) and when visualized at higher magnification in the cortex and hippocampus (scale bars 100 μm). In APP23 mice treated with Adu alone the fluorescent Adu is detected bound to plaques, which were immunolabeled with 4G8 antibody. (B) Levels of Adu are higher when Adu was delivered with SUS in SUS+Adu-treated mice. The plaques of SUS+Adu-treated mice are decorated all over with Adu, whereas with Adu alone the Adu is mainly confined to the outsides of the plaque. Microglia as identified by IBA1 immunostaining are near plaques which have Adu bound to them. (C) Levels of fluorescent antibody in cortical brain lysate is increased in the SUS+Adu group compared to the Adu group. Data is represented as mean±SEM. Statistical differences: *p<0.05. Data were analyzed with t-test. Adu N=9, SUS+Adu N=9.

FIG. 5. Affinity measurement of Aducanumab analog. The affinity of Aducanumab analog for fibrillar amyloid-β42 was measured by ELISA and compared to the antibody 6E10.

FIG. 6. Characterization of microbubbles. In-house prepared microbubbles were analyzed by Coulter Counter.

### DETAILED DESCRIPTION OF THE EMBODIMENTS

It will be understood that the invention disclosed and defined in this specification extends to all alternative combinations of two or more of the subject features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The experimental data generated by the inventors show, amongst other things, an unexpected differential effect with the combination of SUS and an analog of aducanumab (herein referred to as “Adu” or “aducanumab”) (preferably with microbubbles) on the effect of plaque (total vs black plaque vs amber plaque) in the cortex. The Campbell-Switzer stain is the gold standard staining method to give best representation of amyloid distribution and morphology that neither thioflavin staining or anti-Abeta staining can achieve. Specifically, aducanumab delivered by SUS effectively reduced amyloid plaques in the cortex of APP23 mice.

Also unexpectedly, in a behavioural test in a preclinical model of pathogenic amyloid beta protein, a combination of SUS and aducanumab resulted in a significantly greater effect than either SUS alone or aducanumab alone.

In more detail, in the multi-arm study described in the Examples the inventors compared the effects of SUS, aducanumab delivered peripherally and aducanumab delivered to the brain using SUS in APP23 mice with plaque pathology. The inventors found that in their treatment paradigm aducanumab delivered across the blood brain barrier with scanning ultrasound (SUS) markedly reduced amyloid plaque burden in the cortex of 22 month old APP23 mice to an extent greater than that obtained with the antibody or scanning ultrasound alone. In this group, when measured three days post-treatment, aducanumab levels were still 5-fold increased in the combination therapy compared to delivery of aducanumab on its own. Plaque reduction in the hippocampus could be achieved with aducanumab, SUS alone or combined aducanumab and SUS treatment. Spatial memory was improved in 14 month old APP23 mice following four weekly treatments with the combination of aducanumab and SUS. Aducanumab levels in the brain are increased in mice receiving SUS compared to mice receiving aducanumab by peripheral delivery.

General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified,” it is meant that the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding site” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv. Alternatively, the antigen binding domain may be in the context of an entire antibody.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains covalently linked and two light chains. A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is ˜330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html).

The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody.

A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding site or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that an antigen binding site of the invention reacts or associates more frequently, more rapidly, with greater duration and/or with greater affinity with a particular antigen or cell expressing same than it does with alternative antigens or cells.

As used herein, the term “does not detectably bind” shall be understood to mean that an antigen binding site, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using biosensor analysis (e.g. Biacore) in which the antigen binding site is immobilized and contacted with an antigen.

As used herein, the term “does not significantly bind” shall be understood to mean that the level of binding of an antigen binding site of the invention to a polypeptide is not statistically significantly higher than background, e.g., the level of binding signal detected in the absence of the antigen binding site and/or in the presence of a negative control protein (e.g., an isotype control antibody) and/or the level of binding detected in the presence of a negative control polypeptide. The level of binding is detected using biosensor analysis (e.g. Biacore) in which the antigen binding site is immobilized and contacted with an antigen.

As used herein, the term “epitope” (syn. “antigenic determinant”) shall be understood to mean a region of amyloid beta to which an antigen binding site comprising an antigen binding domain of an antibody binds. Unless otherwise defined, this term is not necessarily limited to the specific residues or structure to which the antigen binding site makes contact. For example, this term includes the region spanning amino acids contacted by the antigen binding site and 5-10 (or more) or 2-5 or 1-3 amino acids outside of this region. In some examples, the epitope comprises a series of discontinuous amino acids that are positioned close to one another when antigen binding site is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the term “subject” shall be taken to mean any animal including humans, for example a mammal. Exemplary subjects include but are not limited to humans and non-human primates. For example, the subject is a human.

In any aspect of the present invention, the subject is at least 50, 55, 60, 65, 70, 75, 80, 85, 90 or 95 years old. Preferably, the aged individual is at least 50, 55, 60, 65, 70, 75, 80, 85, 90 or 95 years old. Even more preferably, the aged individual is at least 60, 65, 70, 75, 80, 85, 90 or 95 years old. In any aspect, the subject has cognitive impairment as described herein, for example mild cognitive impairment.

A skilled person will understand that executive functions include a set of cognitive processes that are necessary for the cognitive control of behaviour. Executive functions include basic cognitive processes such as attentional control, cognitive inhibition, inhibitory control, working memory, and cognitive flexibility. Higher order executive functions require the simultaneous use of multiple basic executive functions and include planning and fluid intelligence (i.e., reasoning and problem solving).

A skilled person will understand that a motor skill includes a learned ability to cause a predetermined movement outcome with maximum certainty. Motor learning is the relatively permanent change in the ability to perform a skill as a result of practice or experience. Performance is an act of executing a motor skill. The goal of motor skills is to optimize the ability to perform the skill at the rate of success, precision, and to reduce the energy consumption required for performance. Continuous practice of a specific motor skill will result in a greatly improved performance.

Antibodies

In one example, an antigen binding site or amyloid-beta-binding protein as described herein according to any aspect, embodiment or example is an antibody. Antigen binding sites or binding proteins that binds to amyloid-beta are known in the art, including those described herein. Exemplary Anti-Amyloid-β antibodies are described in van Dyck et al. Biol Psychiatry. 2018 Feb. 15; 83(4): 311-319, the entire contents of that document with reference to those antibodies is hereby incorporated by reference. Aducanumab is one example, along with others including Bapineuzumab, Solanezumab, Gantenerumab, Crenezumab, Ponezumab, and BAN2401.

Methods for generating antibodies are known in the art and/or described in Harlow and Lane (editors) Antibodies: A Laboratory Manual, Cold Spring Harbor Laboratory, (1988). Generally, in such methods amyloid beta (e.g., human amyloid beta) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemistry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present invention may be a synthetic antibody. For example, the antibody is a chimeric antibody, a humanized antibody, a human antibody synhumanized antibody, primatized antibody, a de-immunized antibody or a composite antibody.

Antigen Binding Domain Containing Proteins

Single-Domain Antibodies

In some examples, an antigen binding site is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, Mass.; see, e.g., U.S. Pat. No. 6,248,516).

Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fᵥ) or is absent, and wherein the V_{H} of one polypeptide chain binds to a V_{L} of the other polypeptide chain to form an antigen binding domain, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv)

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the VH and VL of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “V_{HH} domains” in camelid antibodies and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fabs (e.g., as described in EP19930302894).

Mutations to Proteins

The present invention also provides an antigen binding site or a nucleic acid encoding same having at least 80% identity to a sequence disclosed herein. In one example, an antigen binding site or nucleic acid of the invention comprises sequence at least about 85% or 90% or 95% or 97% or 98% or 99% identical to a sequence disclosed herein.

Alternatively, or additionally, the antigen binding site comprises a CDR (e.g., three CDRs) at least about 80% or 85% or 90% or 95% or 97% or 98% or 99% identical to CDR(s) of a VH or VL as described herein according to any example.

In another example, a nucleic acid of the invention comprises a sequence at least about 80% or 85% or 90% or 95% or 97% or 98% or 99% identical to a sequence encoding an antigen binding site having a function as described herein according to any example. The present invention also encompasses nucleic acids encoding an antigen binding site of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding site described herein. A “moderate stringency” is defined herein as being a hybridization and/or washing carried out in 2×SSC buffer, 0.1% (w/v) SDS at a temperature in the range 45° C. to 65° C., or equivalent conditions. A “high stringency” is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5° C. or within 10° C.) or equal to the Tm of a nucleic acid is considered to be high stringency. Medium stringency is to be considered to be within 10° C. to 20° C. or 10° C. to 15° C. of the calculated Tm of the nucleic acid.

The present invention also contemplates mutant forms of an antigen binding site of the invention comprising one or more conservative amino acid substitutions compared to a sequence set forth herein. In some examples, the antigen binding site comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding site comprises 10 or fewer, e.g., 9 or 8 or 7 or 6 or 5 or 4 or 3 or 2 or 1 non-conservative amino acid substitutions.

In one example, the mutation(s) occur within a FR of an antigen binding domain of an antigen binding site of the invention. In another example, the mutation(s) occur within a CDR of an antigen binding site of the invention.

Exemplary methods for producing mutant forms of an antigen binding site include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, N Y, 1995).

Exemplary methods for determining biological activity of the mutant antigen binding sites of the invention will be apparent to the skilled artisan and/or described herein, e.g., antigen binding. For example, methods for determining antigen binding, competitive inhibition of binding, affinity, association, dissociation and therapeutic efficacy are described herein.

Constant Regions

The present invention encompasses antigen binding sites and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG2a constant region.

In one example, the Fc region of the constant region mediates effector function and it has not been mutated to reduce effector function.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG2a constant region. The term “stabilized IgG2a constant region” will be understood to mean an IgG2a constant region that has been modified to reduce Fab arm exchange or the propensity to undergo Fab arm exchange or formation of a half-antibody or a propensity to form a half antibody. “Fab arm exchange” refers to a type of protein modification for human Ig2a, in which an IgG2a heavy chain and attached light chain (half-molecule) is swapped for a heavy-light chain pair from another IgG2a molecule. Thus, IgG2a molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG2a antibody dissociates to form two molecules each containing a single heavy chain and a single light chain.

In one example, a stabilized IgG2a constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

Protein Production

In one example, an antigen binding site described herein according to any example is produced by culturing a hybridoma under conditions sufficient to produce the protein, e.g., as described herein and/or as is known in the art.

Recombinant Expression

In another example, an antigen binding site described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. Nos. 4,816,567 or 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Isolation of Proteins

Methods for isolating a protein are known in the art and/or described herein.

Where an antigen binding site is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding site prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

Assaying Activity of an Antigen Binding Site

Binding to amyloid-beta and Mutants Thereof

It will be apparent to the skilled artisan from the disclosure herein that antigen binding sites as described herein bind to amyloid-beta protein as described herein, or a peptide as described herein. Methods for assessing binding to a protein or peptide are known in the art, e.g., as described in Scopes (In: Protein purification: principles and practice, Third Edition, Springer Verlag, 1994). Such a method generally involves immobilizing the antigen binding site and contacting it with labelled antigen (amyloid-beta). Following washing to remove non-specific bound protein, the amount of label and, as a consequence, bound antigen is detected. Of course, the antigen binding site can be labelled and the antigen immobilized. Panning-type assays can also be used.

Alternatively, or additionally, surface plasmon resonance assays can be used. Optionally, the dissociation constant (Kd), association constant (Ka) and/or affinity constant (K_{D}) of an immobilized antigen binding site for amyloid-beta or an epitope thereof is determined. The “Kd” or “Ka” or “K_{D}” for an amyloid-beta-binding protein is in one example measured by a radiolabelled or fluorescently-labelled amyloid-beta ligand binding assay. In the case of a “Kd”, this assay equilibrates the antigen binding site with a minimal concentration of labeled amyloid-beta or epitope thereof in the presence of a titration series of unlabelled amyloid-beta. Following washing to remove unbound amyloid-beta or epitope thereof, the amount of label is determined, which is indicative of the Kd of the protein.

According to another example the Kd, Ka or K_{D} is measured by using surface plasmon resonance assays, e.g., using BIAcore surface plasmon resonance (BIAcore, Inc., Piscataway, N.J.) with immobilized amyloid beta or a region thereof or immobilized antigen binding site.

Preferably, an antigen binding site as described herein has an EC50 of less than 100 nM, 50 nM, 20 nM, 10 nM, 5 nM, 1 nM, 0.5 nM or 0.2 nM for binding aggregated Abeta1-42. Preferably, an antigen binding site as described herein has an EC50 of less than 100 nM, 50 nM, 20 nM, 10 nM, 5 nM, 1 nM, 0.5 nM, 0.2 nM 0.1 nM for binding fibrillary Abeta1-42. Preferably, an antigen binding site as described herein has an EC50 for of greater than 100 nM, 250 nM, 500 nM or 1 uM for binding to soluble Abeta 1-40. Typically, the antibody is in monoclonal IgG format.

Scanning Ultrasound

As a means to transiently open up the blood brain barrier, the present inventors have found that the application of acoustic energy such as scanning ultrasound (SUS) acts as an effective means for permitting or facilitating the delivery of the antigen binding sites described herein. Application or administration of acoustic energy may permit or facilitate the passage of an antigen binding site through the BBB such that the antigen binding site is then capable of binding to amyloid-beta, preferably in a pathogenic form. Further, application or administration of acoustic energy may permit or facilitate the passage of an antigen binding site through a cell membrane, for example in the cytoplasm of a neuron or a glial cell.

Ultrasound delivery is based on the concept of noninvasive delivery of focused ultrasound pulses that generally comprise a lipid or polymer shell, a stabilized gas core, and a diameter of less than 10 mm. In other words, the acoustic energy, such as ultrasound, can be directed by simple aiming techniques, such as physically orienting one or more transducers on a headpiece, thereby eliminating the complexities of electronic focusing and reduces the need for image guidance. This treatment also has the advantage of treating conditions where the precise site of therapy is not well defined. A highly focused approach is more likely to be unsuccessful or only partially cover the targeted region.

Acoustic energy, such as ultrasound, can be applied to the entire brain or a region of the brain. A region of the brain may be a hemisphere or forebrain. The region may be at least 25% by volume of the brain. The region of the brain may be one that is known to be associated with pathogenic protein deposition such as amyloid beta (Aβ). The particular regions of the brain to be targeted for effective treatment will differ depending on the disease. For example, for Alzheimer's disease the areas that may be targeted include the hippocampus, temporal lobe and/or basal forebrain, more specifically, the hippocampus, mamillary body and dentate gyrus, posterior cingulate gyrus, and temporal lobe. For Frontotemporal Dementia the brain region to be targeted includes the cortex. For Amyotrophic Lateral Sclerosis the region to be targeted includes the spinal cord, motor cortex, brain stem.

Identifying a region of the brain to which acoustic energy is applied may include determining a volume of the brain on the basis of symptoms displayed by the subject, typically clinically observable or biochemically detectable symptoms, or determining a volume of the brain on the basis of a known association with a neurodegenerative disease, in particular those associated with protein oligomers, aggregates or deposits, or determining a volume of the brain including a volume surrounding an site having extracellular protein in a pathogenic form, such as oligomers, an aggregate or deposit.

The focus of the acoustic energy source, typically a scanning ultrasound transducer, may be moved in a pattern with space between the subject sites of application over a region of the brain as described herein or the entire brain. The focus may be moved by a motorised positioning system. In a preferred form, the methods of the invention involve the application of focussed ultrasound to a plurality of locations in the brain. The focussed ultrasound may be applied at 2, 3, 4, 5, 6, 7, 8, 9, 10 or more locations in the brain or on each hemisphere.

It is also contemplated that any disease, condition or syndrome that is a consequence of or associated with aggregation or deposition of amyloid beta proteins in the brain, may be treated by a method of the invention. In addition, a symptom of a disease, condition or syndrome that is a consequence of or associated with aggregation or deposition of proteins in the brain, may be reduced in severity or incidence by a method of the invention.

Increasing the permeability of the blood-brain barrier can be promoted by various agents. These agents are based on the principle that biologically inert and preformed microbubbles, with either a lipid or polymer shell, a stabilized gas core, and a diameter of less than 10 μm, and/or a mean diameter of about 1.9 μm (or other size described herein), can be systemically administered and subsequently exposed to noninvasively delivered focused ultrasound pulses.

In an embodiment of the invention, scanning ultrasound may be combined with microbubbles to disrupt the blood-brain barrier (BBB) which is achieved by mechanical interactions between the microbubbles and the blood vessel wall as pulsed focused ultrasound is applied, resulting in cycles of compression and rarefaction of the microbubbles. This leads to a transient disruption of tight junctions and the uptake of blood-borne factors by the brain. Microbubbles within the target volume become “acoustically activated” by what is known as acoustic cavitation. In this process, the microbubbles expand and contract with acoustic pressure rarefaction and compression over several cycles. This activity has been associated with a range of effects, including the displacement of the vessel wall through dilation and contraction. More specifically, the mechanical interaction between ultrasound, microbubbles, and the vasculature transiently opens tight junctions and facilitates transport across the BBB.

The microbubble agent can be any agent known in the art including lipid-type microspheres or protein-type microspheres or a combination thereof in an injectable suspension. For example, the agent can be selected from the group consisting of Octafluoropropane/Albumin (Optison), a perflutren lipid micro sphere (Definity), Galactose-Palmitic Acid microbubble suspension (Levovist) Air/Albumin (Albunex and Quantison), Air/Palmitic acid (Levovist/SHU508A), Perfluoropropane/Phospholipids (MRX115, DM P115), Dodecafluoropentane/Surfactant (Echogen/QW3600), Perfluorobutane/Albumin (Perfluorocarbon exposed sonicated dextrose albumin), Perfluorocarbon/Surfactant (QW7437), Perfluorohexane/Surfactant (Imagent/AF0150), Sulphur hexafluoride/Phospholipids (Sonovue/BR1), Perfluorobutane/Phospholipids (BR14), Air/Cyanoacrylate (Sonavist/SHU563A), and Perfluorocarbon/Surfactant (Sonazoid/NC100100).

The microbubble agent may be provided as a continuous infusion or as a single bolus dose. A continuous infusion of microbubble, preferably provided over the duration of the acoustic energy application, would be preferred. Typically, the microbubble agent is delivered intravenously through the systemic circulation. For methods of the invention that include the use of an agent such as a microbubble or other cavitation based promotion of blood-brain barrier permeability, the agent may be localized at, or near, or in a region that is targeted with the ultrasound such that the potential of unwanted damage from cavitation effects is minimised.

The applying step, for the delivery of acoustic energy, may comprise the delivery of acoustic energy from an acoustic energy source through a fluid coupler applied directly to the head of the subject. In this application, the fluid coupler may be applied to only one side or aspect of the subject's head. The head may be an unmodified head or a head with a surgically created window in the skull—the fluid coupler being in contact with the window. The acoustic energy may be generated by an unfocused acoustic energy transducer or a phased array acoustic energy transducer (i.e., focused acoustic energy). Significantly, the phased array acoustic energy transducer may be a diagnostic phased array. Diagnostic phased arrays are generally of lower power and are commonly available. The fluid coupler may comprise a contained volume of fluid (e.g., about 50 cc, about 100 cc, about 200 cc, about 400 cc, about 500 cc, about 600 cc or about 1 litre). The fluid may be, for example, water, acoustic energy gel, or a substance of comparable acoustic impedance. The fluid may be contained in a fluid cylinder with at least a flexible end portion that conforms to the subject's head. In other embodiments, the contained volume of fluid may be a flexible or elastic fluid container.

Increased permeability of the blood-brain barrier may be determined by any suitable imaging method. Preferably, the imaging method is MRI, an optical imaging method, positron emission tomography (PET), computerized tomography (CT) or computerized axial tomography (CAT) or ultrasound. If a level of acoustic energy is applied, the increased permeability of the blood-brain barrier could then be determined by any one of the methods described herein and an increased level of acoustic energy could be subsequently applied until the permeability of the bloodbrain barrier had increased to a clinically relevant level. The permeability of the BBB may also be determined by a number of known techniques including injection with Evans blue dye that binds to albumin, a protein that is normally excluded from the brain.

Any ultrasound parameters that result in clinically safe application of acoustic energy are useful in the invention. Typically, the ultrasound parameters that are preferred as those that result in an increase the permeability of the blood-brain barrier, or activate microglia phagocytosis. Various ultrasound parameters can be manipulated to influence the permeability increase in the blood-brain barrier and these include pressure amplitude, ultrasound frequency, burst length, pulse repetition frequency, focal spot size and focal depth. Several parameters are now described that are useful in a method of the invention.

Focal spot size useful in a method of the invention includes about a 1 mm to 2 cm axial width. Typically, the focal spot size has an axial width of about 1 mm to 1.5 cm, preferably 1 mm to 1 cm, even more preferably 1 mm to 0.5 cm. The length of the focal spot may be about 1 cm to as much as about 15 cm, preferably 1 cm to 10 cm, even ore preferably 1 cm to 5 cm. The focal size useful in a method of the invention is one that allows an increase in the permeability of the blood-brain barrier of the subject.

The focal depth of the ultrasound generally depends on the areas of the brain affected by the disease. Therefore, the maximum focal depth would be the measurement from the top of the brain to the base, or about 10 to about 20 cm. Focal depth could be altered by electronic focusing, preferably by using an annular array transducer. The focal depth allows application to the cortical layer which, for example, may be up to 4 cm deep.

Typically the ultrasound is applied in continuous wave, burst mode, or pulsed ultrasound. Preferably the ultrasound is applied in burst mode, or pulsed ultrasound. Pulse length parameters that are useful in a method the invention include between about 1 to about 100 milliseconds, preferably the pulse length or burst length is about 1 to about 20 milliseconds. Exemplary burst mode repetition frequencies can be between about 0.1 to 10 Hz, 10 Hz to 100 kHz, 10 Hz to 1 kHz, 10 Hz to 500 Hz or 10 Hz to 100 Hz.

The duty cycle (% time the ultrasound is applied over the time) is given by the equation duty cycle=pulse length×pulse repetition frequency×100. Typically, the duty cycle is from about 0.1% to about 50%, about 1% to about 20%, about 1% to about 10%, or about 1% to about 5%.

The ultrasound pressure useful in a method of the invention is the minimum required to increase the permeability of the blood-brain barrier. The human skull attenuates the pressure waves of the ultrasound which also depends on the centre frequency of the transducer, with lower centre frequencies of the ultrasound transducer causing better propagation and less attenuation. A non-limiting example of ultrasound pressure is between 0.1 MPa to 3 MPa, preferably about 0.4 or 0.5 MPa. Typically this pressure is applied to the skull, i.e transcranially. The mechanical index characterises the relationship between peak negative pressure amplitude in situ and centre frequency with mechanical index=Pressure (MPa)/sqrt centre frequency (MHz) if this mechanical index was free from attenuation/measured from within the skull, the mechanical index would be between about 0.1 and about 2, preferably about 0.1 to 1 or 0.1 to 0.5.

A non-limiting example of a system that is able to open the blood-brain barrier is the TIPS system (Philips Research). It consists of a focused ultrasound transducer that generates a focused ultrasound beam with a centre frequency of 1-1.7 MHz focal depth of 80 mm, active outer diameter 80 mm, active inner diameter 33.5 mm which is driven by a programmable acoustic signal source within the console and attached to a precision motion assembly. An additional example of a system that is able to generate an ultrasound beam suitable for blood-brain barrier disruption is the ExAblate Neuro (Insightec) system. Suitable parameters for blood-brain barrier opening in humans such as centre frequency and microbubble dosage may be different to that in mice.

For any of the method or apparatus of the invention, the ultrasound transducer may have an output frequency of between 0.1 to 10 MHz, or 0.1 to 2 MHz. The ultrasound may be applied for a time between 10 milliseconds to 10 minutes. The ultrasound may be applied continuously or in a burst mode.

Image contrast agents, used in any methods of the invention, may be selected from the group consisting of magnetic resonance contrast agents, x-ray contrast agents (and x-ray computed tomography), optical contrast agents, positron emission tomography (PET) contrast agents, single photon emission computer tomography (SPECT) contrast agents, or molecular imaging agents. For example, the imaging contrast agent may be selected from the group consisting of gadopentetate dimeglumine, Gadodiamide, Gadoteridol, gadobenate dimeglumine, gadoversetamide, iopromide, lopam idol, Ioversol, or Iodixanol, and lobitridol.

The frequency of application of the ultrasound would generally depend on patient severity. The parameters of the ultrasound and the treatment repetition are such that there is an increase in permeability of the blood-brain barrier but preferably wherein there is no, or clinically acceptable levels of, damage to parenchymal cells such as endothelial or neuronal damage, red blood cell extravasation, haemorrhage, heating and/or brain swelling. Any method of the invention may further include performing magnetic resonance imaging on a subject comprising the steps of (a) administering a magnetic resonance contrast agent to a subject through the blood-brain barrier using any of the methods of the invention and performing magnetic resonance imaging on said subject. In this context the use of magnetic resonance imaging is to confirm the increase in permeability of the blood-brain barrier and not to locate the presence of a pathogenic protein.

Another embodiment of the invention involves providing an imaging contrast agent to the whole brain including the steps of administering an imaging contrast agent into the bloodstream of said subject; and applying ultrasound to the brain of said subject to open the bloodbrain barrier to allow the imaging contrast agent to cross the blood-brain barrier. The imaging contrast agent can be administered to the subject simultaneously or sequentially with the application of the ultrasound. In this embodiment the sequential administration of the contrast agent can be prior to or post application of the ultrasound such as SUS. In a preferred embodiment, any of the agents described herein may be administered to the bloodstream between 0 to 4 hours, between 2 to 4 hours or between 3-4 hours after ultrasound treatment using one of the methods of the invention. Preferably, the agents described herein are co-delivered.

Conditions to be Treated

The antigen binding sites of the present invention are useful in the treatment or prevention of any condition associated, or caused by, the presence, over-expression or accumulation of amyloid-beta, also referred to as amyloid-beta deposits, aggregates or plaques herein.

Amyloid beta (Aβ) denotes peptides of 36-43 amino acids and is the main component of amyloid plaques found in the brains of Alzheimer patients. Amyloid beta is also present in pre-amyloid parenchymal deposits and within the walls of leptomeningeal and cerebral vessels. Amyloid beta is composed of 11-15 amino acids of the transmembrane domain and 28 amino acids of the extracellular domain of amyloid precursor protein (APP). The functions of APP in the nervous system include mediating adhesion and in the growth of neuronal and non-neuronal cells.

APP is cleaved by beta secretase and gamma secretase to yield Aβ. APP occurs as several Aβ-containing isoforms of 695, 751, and 770 amino acids, with the latter two APP containing a domain that shares structural and functional homologies with Kunitz serine protease inhibitors. Amyloid beta molecules can aggregate to form flexible soluble oligomers which may exist in several forms. It is believed that certain misfolded oligomers can induce other Aβ molecules to also take the misfolded oligomeric form. These oligomers, as well as some folded forms, are toxic to nerve cells.

The term “amyloid beta” or “Aβ” as provided herein includes homologues or variants that, in a non-disease form, maintain the activity of amyloid beta (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

In some embodiments, the amyloid beta protein and peptides derived from amyloid precursor protein may be as identified by the UniProt sequence reference UniProtKB—P05067 (A4_HUMAN), homolog or functional fragment thereof.

Amyloid beta (herein also referred to as Aβ) is formed after sequential cleavage of the amyloid precursor protein (APP), a transmembrane glycoprotein of undetermined function. APP can be cleaved by the proteolytic enzymes α-, β- and γ-secretase; Aβ protein is generated by successive action of the β and γ secretases. The γ secretase, which produces the C-terminal end of the Aβ peptide, cleaves within the transmembrane region of APP and can generate a number of isoforms of 30-51 amino acid residues in length. The most common isoforms are Aβ40 and Aβ42; the longer form is typically produced by cleavage that occurs in the endoplasmic reticulum, while the shorter form is produced by cleavage in the trans-Golgi network. The Aβ40 form is the more common of the two, but Aβ42 is the more fibrillogenic and is thus associated with disease states. Mutations in APP associated with early-onset Alzheimer's disease have been noted to increase the relative production of Aβ42, and thus one suggested avenue of Alzheimer's disease therapy involves modulating the activity of β- and γ-secretases to produce mainly Aβ40.

In Alzheimer's disease other neurodegenerative diseases, the deposition of aggregates enriched in amyloid-beta has been reported. When misfolded, this otherwise very soluble protein can form extremely insoluble aggregates that contribute to a number of pathologies. Accumulating Aβ deposits may result in Aβ oligomerization, gradually leading to deposits in the form of fibrils and plaques. One form, beta-amyloid 42, is thought to be especially toxic. In Alzheimer's, abnormal levels of this naturally occurring protein clump together to form plaques that collect between neurons and disrupt cell function.

A subject in need of treatment may be one that exhibits impaired memory function, cognitive function or subclinical or clinical symptoms of a neurodegenerative disease. The selection of a subject for treatment may involve a screening step for identifying whether the subject is displaying impaired cognitive function, memory function or a clinical manifestation of a neurodegenerative disease. A subject in need of treatment may be one that is identified as having early, intermediate or late stage disease and in the case of Alzheimer's disease may be identified as having either diffuse Aβ oligomers or plaques.

At a clinical level, Alzheimer's disease may present a number of cognitive symptoms including mental decline, difficulty thinking and understanding, depression, hallucination, or paranoia, confusion in the evening hours, delusion, disorientation, forgetfulness, making things up, mental confusion, difficulty concentrating, inability to create new memories, inability to do simple maths, or inability to recognise common things. Behavioural symptoms may also be present and include aggression, agitation, difficulty with self-care, irritability, meaningless repetition of own words, personality changes, lack of restraint, or wandering and getting lost. Loss of loss of appetite or restlessness may also be present.

Thus, when a patient presents to a doctor with any of the above symptoms, some of the commonly used diagnostic tests include cognitive tests. Cognitive tests are used to measure and evaluate cognitive, or ‘thinking’, functions such as memory, concentration, visual-spatial awareness, problem solving, counting and language skills. Particular cognitive tests that may be used include the following.

Mini-Mental Status Examination (MMSE)

The MMSE is the most common test for the screening of dementia. It assesses skills such as reading, writing, orientation and short-term memory.

Alzheimer's Disease Assessment Scale-Cognitive (ADAS-Cog)

This 11-part test is more thorough than the MMSE and can be used for people with mild symptoms. It is considered the best brief examination for memory and language skills.

Neuropsychological Testing

A variety of tests will be used and may include tests of memory such as recall of a paragraph, tests of the ability to copy drawings or figures and tests of reasoning and comprehension.

Brain Imaging Techniques

Various brain-imaging techniques are sometimes used to show brain changes and to rule out other conditions such as tumour, infarcts (strokes—dead areas of brain tissue) and hydrocephalus (fluid on the brain); these include:

(a) Computed Tomography (CT or CAT) Scan

This technique involves taking many X-rays from different angles in a very short period of time. These images are then used to create a 3-dimensional image of the brain. CT scans are mainly used to rule out other causes of dementia such as stroke, brain tumour, multiple sclerosis or haemorrhage. They can show certain changes that are characteristic of Alzheimer's disease or other causes of dementia.

(b) Magnetic Resonance Imaging (MRI)

This technique uses powerful magnets and radiowaves to produce very clear 3-dimensional images of the brain. Currently MRI is the radiological test of choice. As well as ruling out treatable causes of dementia, MRI can reveal patterns of brain tissue loss, which can be used to discriminate between different forms of dementia such as Alzheimer's disease and frontotemporal dementia.

(c) Positron Emission Tomography (PET) and Single-Photon Emission Computerized Tomography (SPECT)

In both of these tests, a small amount of radioactive material is injected into the patient and detectors in the scanner detect emissions from the brain. PET provides visual images of activity in the brain. SPECT is used to measure blood flow to various regions of the brain.

A patient with frontotemporal dementia may show impairments in one or more of the domains of language, social cognition, perceptual-motor, executive function and complex attention without learning and memory impairment, or learning and memory impairment may be present. In Parkinson's disease motor deficits may be present with or without deficits in other domains of cognition, or deficits may be present. In Huntington's disease, motor deficits may be present without deficits in other domains of cognition, or deficits may be present. In Amyotrophic Lateral Sclerosis motor deficits may be present without deficits in other domains of cognition, or deficits may be present.

The neurodegenerative diseases to which the invention can be applied are those where pathogenic protein is extracellular and causes or contributes to the disease or a symptom thereof. The pathogenic protein may be in pathogenic form when in an altered structure such as an oligomer, an aggregate or a deposit. Alzheimer's disease, dementia with Lewy bodies, Parkinson's disease, frontotemporal lobar degeneration and British and Danish familial dementia are non-limiting examples of diseases associated with extracellular pathogenic protein. Alzheimer's disease is the most common example of these diseases in which oligomers or plaques composed of amyloid beta (Aβ) are formed in the brain. Other neurodegenerative diseases are caused by the pathological aggregation of one or more of the proteins: Amyloid beta (Aβ), amyloid fragments, amyloid precursor protein, amyloid precursor protein fragments or British peptide.

In a preferable embodiment the condition, disease or syndrome is Alzheimer's disease. In this case the subject to be treated may display impairment in the following cognitive domains including learning and memory, complex attention, executive function, perceptual motor, social cognition, and language. Alternatively, the subject may display one or more of the following symptoms: Age-associated cognitive impairment, Age-associated neuronal dysfunction not restricted to cognitive impairment, short term memory loss, inability to acquire new information, semantic memory impairments, apathy, mild cognitive impairment, language, executive or visuoconstructional problems or apraxia, long term memory impairment, irritability and aggression, and exhaustion.

Treatment as used herein refers to therapeutic treatment and also involves ameliorating a symptom associated with a disease. Therapeutic treatment can be measured by an increase or recovery in any one or more of the group consisting of cognitive function; short term memory; ability to acquire new information; semantic memory; apathy; language, executive or visuoconstructional problems or apraxia; long term memory; irritability and aggression; or exhaustion. Treatment can also be measured via reduction in the presence of pathogenic protein or a reduction in the particular forms of pathogenic protein such as protein aggregates or deposits. The presence and reduction of the pathogenic protein that can be visualised or detected by imaging techniques or biochemical techniques described herein. For example, in relation to Alzheimer's disease, treatment may relate to a reduction in a soluble or insoluble isoforms of amyloid beta (Aβ) peptide or a reduction in the number of amyloid beta (Aβ) plaques. Alternatively, the outcome of the treatment may be determined by neuropsychological or cognitive testing.

Improving memory may be determined by memory tests, typically a test administered by a clinical professional. Standardised neuropsychological tests of cognition that could be administered to test the effectiveness of the treatment include any of the following tests or one or more of its components: Neuropsychological Test Battery, Alzheimer's Disease Assessment Scale-cognitive sub scale (ADAS-cog), Mini-Mental State Examination, Severe Impairment Battery, Disability Assessment Scale for Dementia, Clinical Dementia Rating Scale Sum of Boxes, Alzheimer's Disease Cooperative Study Clinical Global Impression of Change, Wechsler Memory Scale Visual Immediate, Wechsler Memory Scale Verbal Immediate, Rey Auditory Verbal Learning Test, Wechsler Memory Digit Span, Controlled Word Association Test, Category Fluency Test, Wechsler Memory Scale Visual Delayed, Wechsler Memory Scale Verbal Delayed, Rey Auditory Verbal Learning Test, Wechsler Memory Scale, Stroop Task, Wisconsin Card Sorting Task, Trail Making Test, or any other tests of memory and executive function alone or in combination.

Various in vitro assays are also known in the art for assessing the ability of an antigen binding site to inhibit or reduce amyloid-beta accumulation leading to a functional response. Assays for assessing therapeutic efficacy are described hereinabove in relation to determining neutralization by an antigen binding site, particularly in Example 1.

To determine whether an antigen binding site and/or SUS of the present invention reduces or inhibits the accumulation of amyloid beta deposits or plaques in mouse models of disease, silver staining may be used (eg Campbell Switzer silver staining). Further, an amyloid beta antibody may be used to quantify levels of amyloid beta following administration of antigen binding site and/or SUS.

In an embodiment, the antigen binding site can be tested in a model of Alzheimer's disease. In this embodiment, a reduction in amyloid beta pathology such as decreases to the accumulation of amyloid beta can be assessed by measuring pathogenic protein load by immunohistochemistry or any method or assay described herein.

Further, for animal models robust behavioural tests may also be conducted to determine improvements in behavioural ability in response to an antigen binding site of the present invention. For instance, the APA test may be conducted, which is a test of hippocampus dependent spatial learning in which mice learned to avoid a shock zone in a rotating arena. Additional tests include a novel object recognition (NOR) test.

Absence of brain damage may be determined by Evans Blue extravasation, absence of edemas, erythrocyte extravasation and ‘dark’ neurons as determined by Nissl staining, hematoxylin and eosin staining to determine the integrity of the cortex and the hippocampus, and absence of ischemic damage using acid fuchsin staining.

Compositions

In some examples, an antigen binding site as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding site into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding site of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

WO2002/080967 describes compositions and methods for administering aerosolized compositions comprising antibodies for the treatment of, e.g., asthma, which are also suitable for administration of an antigen binding site of the present invention.

Dosages and Timing of Administration

Suitable dosages of an antigen binding site of the present invention will vary depending on the specific an antigen binding site, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma may be measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

Kits

The present invention additionally comprises a kit comprising one or more of the following:
(i) an antigen binding site as described herein or expression construct(s) encoding same; and
(ii) a source of acoustic energy, preferably scanning ultrasound (SUS).

In the case of a kit for detecting amyloid-beta, the kit can additionally comprise a detection means, e.g., linked to an antigen binding site of the invention.

In the case of a kit for therapeutic/prophylactic use, the kit can additionally comprise a pharmaceutically acceptable carrier.

Optionally a kit of the invention is packaged with instructions for use in a method described herein according to any example.

TABLE 1
Amino acid and nucleotide sequences (the amino acid sequences of Aducanumab,
also referred to as BIIB037 or NI-101.12F6A, are present in WO2014089500
and WO2008081008. The amino acid and nucleotide sequences from these
publications for BHB037 or NI-101.12F6A are incorporated herein by reference).

| Antibody or | | SEQ | |
|---|---|---|---|
| protein ID | Region | ID NO: | Amino acid or nucleotide sequence |
| Aducanumab | HCDR1 | 1 | SYGMH |
| | HCDR2 | 2 | VIWFDGTKKYYTDSVKG |
| | HCDR3 | 3 | DRGIGARRGPYYMDV |
| | LCDR1 | 4 | RASQSISSYLN |
| | LCDR2 | 5 | AASSLQS |
| | LCDR3 | 6 | QQSYSTPLT |
| | HFR1 | 7 | QVQLVESGGGVVQPGRSLRLSCAASGFAFS |
| | HFR2 | 8 | WVRQAPGKGLEWVA |
| | HFR3 | 9 | RFTISRDNSKNTLYLQMNTLRAEDTAVYYCAR |
| | HFR4 | 10 | WGKGTTVTVSS |
| | LFR1 | 11 | DIQMTQSPSSLSASVGDRVTITC |
| | LFR2 | 12 | WYQQKPGKAPKLLIY |
| | LFR3 | 13 | GVPSRFSGSGSGTDFTLTISSLQPEDFATYYC |
| | LFR4 | 14 | FGGGTKVEIKR |
| | VH | 15 | QVQLVESGGGVVQPGRSLRLSCAASGFAFSSYGMHWVRQAPGKGLEWVAVIWFDG |
| | | | TKKYYTDSVKGRFTISRDNSKNTLYLQMNTLRAEDTAVYYCARDRGIGARRGPYY |
| | | | MDVWGKGTTVTVSS |
| | VL | 16 | DIQMTQSPSSLSASVGDRVTITCRASQSISSYLNWYQQKPGKAPKLLIYAASSLQ |
| | | | SGVPSRFSGSGSGTDFTLTISSLQPEDFATYYCQQSYSTPLTFGGGTKVEIKR |
| | VH (DNA) | 17 | CAGGTGCAGCTGGTGGAGTCCGGAGGAGGAGTGGTGCAGCCAGGCCGGTCCCTGA |
| | | | GACTGTCTTGTGCCGCCAGCGGCTTCGCCTTTAGCTCCTATGGCATGCACTGGGT |
| | | | GAGGCAGGCCCCCGGCAAGGGCCTGGAGTGGGTGGCCGTGATCTGGTTCGACGGC |
| | | | ACCAAGAAGTACTATACAGATAGCGTGAAGGGCAGGTTTACCATCTCCCGCGACA |
| | | | ACTCTAAGAATACACTGTACCTCCAGATGAATACCCTGCGCGCCGAGGACACAGC |
| | | | CGTGTACTATTGCGCCAGGGATCGCGGAATCGGCGCCAGAAGAGGACCTTACTAT |
| | | | ATGGACGTGTGGGGCAAGGGCACCACAGTGACCGTGTCTAGC |
| | VL (DNA) | 18 | GACATCCAGATGACCCAGAGCCCAAGCTCCCTGTCTGCAAGTGTGGGCGATCGGG |
| | | | TCACCATCACATGTAGAGCCTCACAGAGCATTTCTAGTTACCTGAACTGGTATCA |
| | | | GCAGAAGCCAGGGAAAGCTCCCAAGCTGCTGATCTACGCCGCTTCAAGCCTGCAG |
| | | | TCTGGAGTGCCCAGTCGATTCTCCGGCTCTGGGAGTGGAACAGACTTTACTCTGA |
| | | | CCATTTCCTCTCTGCAGCCTGAGGATTTCGCCACTTACTATTGCCAGCAGTCATA |
| | | | TAGCACCCCACTGACATTTGGCGGGGGAACAAAAGTGGAGATCAAG |

### EXAMPLES

Materials and Methods

Animal Model

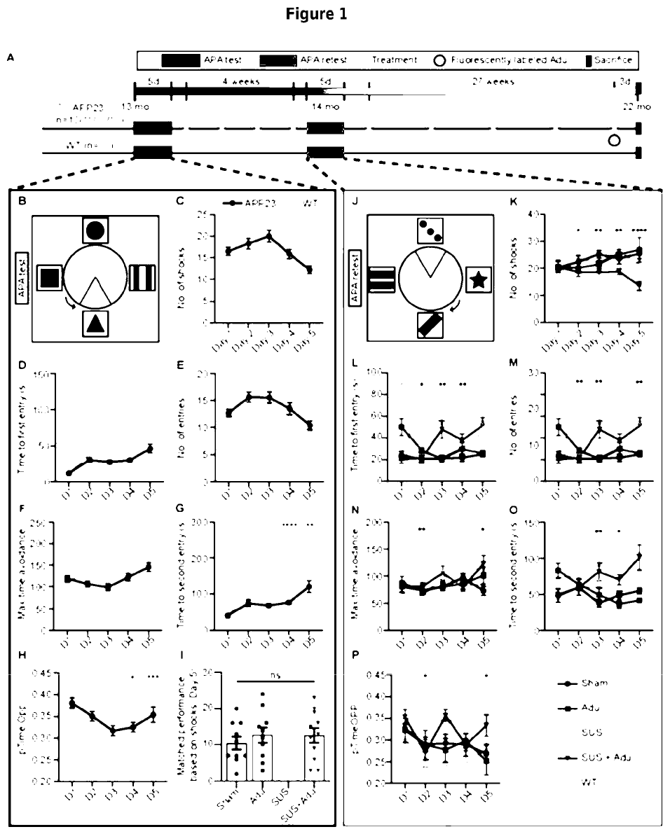

APP23 mice express hAPP751 with the Swedish double mutation under control of the mThy1.2 promoter. APP23 mice are characterized by amyloid plaque formation mainly in the cortex, as well as associated memory deficits, and cerebral amyloid angiopathy as they age. Animal experimentation was approved by the Animal Ethics Committee of the University of Queensland (approval QBI/554/17). In this study, APP23 mice, aged 13 months, were assigned to four treatment groups: sham (N=10), SUS (N=11), Adu (5 mg/kg delivered retroorbitally, N=11), or SUS+Adu (5 mg/kg retroorbitally, N=10). Assignment to treatment groups was based on matching performance of spatial memory (number of shocks) on day 5 of the active place avoidance (APA) test. A group of wild-type mice (N=12) were included. APP23 mice were ranked from those receiving the fewest shocks to those receiving the most shocks on day 5 and were assigned to the four treatment groups (sham, SUS, Adu, SUS+Adu) in rank order. Each group received a total of nine treatments (an APA retest was performed after the forth treatment), with the final treatment in the SUS, and SUS+Adu groups using fluorescently labelled antibody (2.5 mg/kg Alexa Fluor 647-labeled Adu and 2.5 mg/kg unlabeled Adu). (FIG. 1A). Three days after the final treatment, the mice were administered an overdose of sodium pentobarbitone and perfused with phosphate buffered saline (PBS). The right hemisphere of the brain was fixed in paraformaldehyde solution for histology, while the cortex and hippocampus of the left hemisphere were dissected and frozen in liquid nitrogen for subsequent analysis. Due to the increased mortality of this strain, the numbers of mice surviving to 22 months for histological and biochemical analysis were N=10 sham, N=9 Adu, N=8 SUS, N=9 SUS+Adu. Assessment of outcomes was performed with the researcher blinded to the treatment group. All animal experimentation was approved by the Animal Ethics Committee of the University of Queensland (approval number QBI/554/17). Sample sizes for the experiment were selected based on Leinenga, G. and J. Gotz, Sci Transl Med, 2015. 7(278). Data was collected for all mice that survived until the end of the experiment and all data was included.

SUS Equipment

An integrated focused ultrasound system was used (Therapy Imaging Probe System, TIPS, Philips Research). The system consisted of an annular array transducer with a natural focus of 80 mm, a radius of curvature of 80 mm, a spherical shell of 80 mm with a central opening of 31 mm diameter, a 3D positioning system, and a programmable motorized system to move the ultrasound focus in the x and y planes to cover the entire brain area. A coupler mounted to the transducer was filled with degassed water and placed on the head of the mouse with ultrasound gel for coupling, to ensure propagation of the ultrasound to the brain. The focal zone of the array was an ellipse of approximately 1.5 mm×1.5 mm×12 mm.

Production of Microbubbles

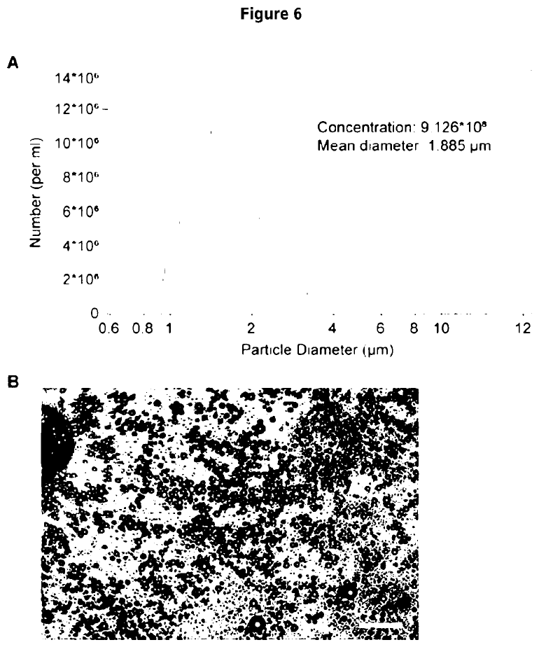

In-house prepared microbubbles comprising a phospholipid shell and octafluoropropane gas core were used. 1,2-distearoyl-sn-glycero-3-phosphocholine (DSPC) and 1,2-distearoyl-sn-glycero-3-phosphoethanolamine-N-[amino(polyethylene glycol)-2000] (DSPE-PEG2000) (Avanti Polar Lipids) were mixed at a 9:1 molar ratio dissolved in chloroform (Sigma) and the chloroform solvent was evaporated under vacuum. The dried phospholipid cake was then dissolved in PBS with 10% glycerol to a concentration of 1 mg lipid/ml and heated to 55° C. in a sonicating water bath. The solution was placed in 1.5 ml glass HPLC vials and the air in the vial was replaced with octafluoropropane (Arcadophta). Microbubbles were generated on the day of the experiment by agitation in a dental amalgamator at 4000 rpm for 40 sec. Activated microbubbles were measured with a Multisizer 4e coulter counter which reported under 10 μm, a mean diameter of 1.885 μm, and a concentration of 9.12×10⁸ microbubbles/ml. These microbubbles were also observed to be polydisperse under a microscope (FIG. 6).

SUS Application

Mice were anesthetized with ketamine (90 mg/kg) and xylazine (6 mg/kg) and the hair on the head was shaved and depilated. Mice were injected retro-orbitally with 1 μl/g body weight of microbubble solution and then placed under the ultrasound transducer with the head immobilized. Parameters for the ultrasound delivery were 1 MHz center frequency, 0.7 MPa peak rarefactional pressure, 10 Hz pulse repetition frequency, 10% duty cycle, and a 6 sec sonication time per spot. The focus of the transducer had dimensions of 1.5 mm×12 mm in the transverse and axial planes, respectively. The motorized positioning system moved the focus of the transducer array in a grid with 1.5 mm spacing between individual sites of sonication so that ultrasound was delivered sequentially to the entire brain. Mice typically received a total of 24 spots of sonication in a 6×4 raster grid pattern. For sham treatment, mice received all injections and were placed under the ultrasound transducer, but no ultrasound was emitted. When the animals were treated with Adu antibody, the solution was mixed briefly with the microbubble solution and injected into the retro-orbital sinus before the mouse was placed under the ultrasound transducer. The time between injecting microbubbles and commencing ultrasound delivery was 60±10 s and the duration of sonication was approximately 3 min (total time from microbubble injection approximately 4 min).

Aducanumab Production

VH and VL sequences were identified in Biogen Idec's patent submission for BUB-037 WO2014089500 A1 and were cloned into mouse IgG2a and kappa pcDNA3.1 vectors (Genscript). Murine chimeric aducanumab (also referred to herein as “Adu”, “Aducanumab analogue/analog” or simply “aducanumab”) was produced using the Expi293 expression system and purified using protein A chromatography and was verified to be endotoxin-free by LAL assay.

Antibody Affinity ELISA

EC50 of murine chimeric aducanumab/Adu was determined by direct-binding ELISA. A131-42 fibrils were generated by incubating 0.1 mM A131-42 (JPT Peptide Technologies, Germany) in 10 mM HCl for 3 d at 37° C. A MaxiSorp ELISA plate was coated with 2p/ml Aβ₁₋₄₂ fibrils in 0.1M sodium bicarbonate buffer and then blocked with 1% bovine serum albumin. The concentration of antibody giving half-maximal binding (EC50) was determined by incubating wells with serial dilutions of aducanumab antibody followed by washing and detecting bound aducanumab with anti-mouse HRP antibody (Dako) and developing with 3,3′,5,5′-tetramethylbenzidine substrate. 6E10 antibody was used as a positive control for Aβ binding and its EC50 was determined for comparison with aducanumab.

Antibody Labelling

Chimeric aducanumab was covalently conjugated with Alexa Fluor 647 dye (Thermo Fisher Scientific) in PBS with 0.1 M sodium bicarbonate as previously described (Nisbet et al 2017, Brain, 2017. 140(5): p. 1220-1230). protein concentration and degree of labelling was determined by measuring absorbance at 280 and 650 nm.

Tissue Processing.

Mice were deeply anesthetized with pentobarbitone before being perfused with 30 ml of phosphate-buffered saline (PBS), and brains were dissected out. One hemisphere of the brain was fixed overnight in a solution of 4% wt/vol paraformaldehyde, then cryoprotected in 30% sucrose and sectioned coronally at 40 μm thickness on a freezing-sliding microtome (SM2000R, Leica). A one-in-eight series of sections was stored in PBS with sodium azide at 4° C. until staining.

Assessment of Amyloid Plaques

For the assessment of amyloid plaque load, an entire one-in-six series of coronal brain sections of one hemisphere at 40 μm was stained using the Campbell-Switzer silver stain protocol that discriminates fibrillar from less aggregated using protocol available from http://neuroscienceassociates.com/Documents/Publications/campbell-switzer_protocol.htm or Leinenga, G. and J. Gotz, Sci Transl Med, 2015. 7(278): p. 278ra33). Stained sections were mounted onto microscope slides and imaged at a 10× magnification on a Metafer bright-field VSlide scanner (MetaSystems) using Zeiss Axio Imager Z2. Amyloid plaque load analysis was performed on all stained sections using ImageJ. A region-of-interest was drawn around the cortex and dorsal hippocampus separately and measured to give the area. As both black and amber plaques are present in the sections and represent different types of amyloid compactness, they were analyzed separately using color deconvolution method and automated thresholding to distinguish the two types of amyloid plaques. For the analysis of black plaques, a color deconvolution vector where R=0.65, G=0.70, B=0.29 was used followed by the MaxEntropy auto thresholding function in ImageJ. As black plaques consist mainly of diffuse fibrils no size filter was applied. To measure amber plaques, a color deconvolution vector where R=0.64, G=0.72 and B=0.27 was used, followed by invert function and automated thresholding using the triangle method in ImageJ, fill-holes function and a 60 μm² size filter was applied. Using this method, plaque number, total plaque area, average plaque size, and % area covered by plaque were obtained for both the black and amber plaques and summed to give total plaque area for the cortex and hippocampus. We were unable to analyze the hippocampus of one mouse in Adu-treated group because of folds in the tissue.

Assessment of Cerebral Amyloid Angiopathy

To assess CAA, a one-in-eight series of Campbell-Switzer silver-stained sections were examined. Regions of interest were drawn manually around areas of CAA in the cortex, which were distinguished from plaques by having a rod-like structure indicative of blood vessels and a diameter greater than 15 μm. Meningeal CAA which has a ring-shaped structure and occurred close to the edge of the section was also measured. The number of CAA deposits per section, the average size and the % area of brain section positive for CAA staining were determined.

Immunofluorescence

Coronal 40 μm sections were co-stained with the 4G8 antibody against Aβ (1:1,000, Covance) and Iba1 (1:1,000 Wako) followed by goat anti-mouse and goat anti-rabbit Alexa Fluor-conjugated secondary antibodies (Thermo Fisher). Alexa Fluor 647-conjugated Adu was detected in situ without additional amplification. Sections were cover-slipped and imaged with a fluorescence slide scanner (Metafer).

Enzyme-Linked Immunosorbent Assay for Amyloid-Beta

Frozen cortices were homogenized in 10 volumes of a solution containing 50 mM NaCl, 0.2% diethylamine (DEA) with complete protease inhibitors, and dounce homogenised by passing through 19 and 27 gauge needles. Samples were then centrifuged at 21,000×g for 90 min at 4° C. The supernatant was retained as the DEA-extracted soluble Aβ fraction. The remaining pellets were resuspended in 10 volumes of 5 M Guanidine HCl, sonicated, and centrifuged at 21,000×g for 30 min at 4° C. The resultant supernatant was retained as the guanidine-extracted insoluble Aβ fraction. The concentrations of Aβ₄₀ and Aβ₄₂ were determined in brain lysates using the ELISA kits according to the manufacturer's instructions (human Aβ₄₀ and Aβ₄₂ brain ELISA, Merck).

Active Place Avoidance test

The APA task is a test of hippocampus-dependent spatial learning. Mice (APP23 mice and non-transgenic littermate controls) were tested over six days in a rotating elevated arena (Bio-Signal group) that had a grid floor and a 32 cm high clear plastic circular fence enclosing a total diameter of grid of 77 cm. High-contrast visual cues were present on the walls of the testing room. The arena and floor was rotated at a speed of 0.75 rpm and a 500 ms, 60 Hz, 0.5 mA mild shock was delivered through the grid floor when the animal entered a 60 degree shock zone, and every 1,500 ms until the animal left the shock zone. The shock zone was maintained at a constant position in relation to the room. Recorded tracks were analyzed with Track Analysis software (Bio-Signal group). A habituation session was performed 24 h before the first training session in which animals were placed in the rotating arena for 5 min to explore but the mice did not receive any shocks during this period. After this initial testing APP23 mice were divided into four groups with mice matched so that the performance (number of shocks) of the four groups of mice on day five of the task was the same. Five training sessions were held on consecutive days, one per day with a duration of 10 min. Following a 4 week-period of treatment in which the mice were given weekly treatments, they were retested in the task (reversal learning). For retesting the shock zone was switched to the opposite side of the arena, the platform was rotated clockwise rather than counterclockwise, and the visual cues were changed but mice were tested in the same room. The number of shocks, numbers of entries, time to first entry, time to second entry, proportion of time spent in the opposite quadrant of the shock zone for sham, SUS, Adu, and SUS+Adu-treated groups were compared over the days of testing.

Statistical Analysis

Statistical analyses were conducted with Prism 8 software (GraphPad). Values were always reported as means±SEM. One-way ANOVA followed by the Holm-Sidak multiple comparisons test, or t-test was used for all comparisons except APA analyses where two-way ANOVA with day as a repeated measures factor and group as a between subjects factor was performed and was followed by the Holm-Sidak multiple comparisons test for simple effects to compare group performance on different days. The model assumption of equal variances was tested by Brown-Forsyth or Bartlett tests, and the assumption of normality was tested by Kolmgorov-Smirnov tests and by inspecting residuals with QQ plots. All observations were independent, with allocation to groups based on active place avoidance where mice were ranked on performance and assigned to one of the four groups (sham, sus, adu, adu+sus) in order of number of shocks on day 5 listed from most to least shocks.

Generation of Aducanumab Analog and Application

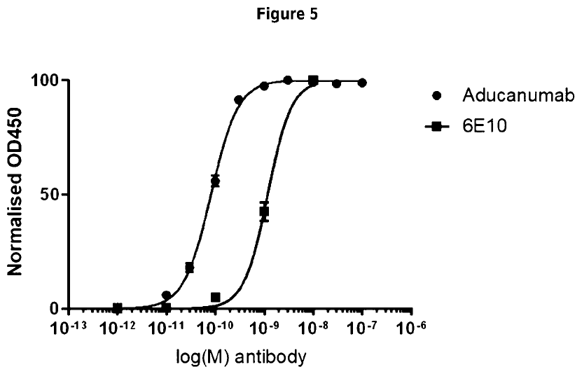

Adu was generated by grafting the VH and VL chains of Aducanumab onto a mouse IgG backbone and expressing Adu in Expi293 cells. We then established that the affinity of Adu to fibrillar A1342 (EC₅₀ 81.7 pM) was similar to that published earlier for Aducanumab (EC₅₀ 100 pM) (Sevigny, J., et al., Nature, 2016. 537(7618): p. 50-6). In comparison, the 6E10 antibody had an EC50 of 1.18 nM for Aβ₄₂ fibrils (FIG. 5). 13-month old APP23 mice were divided into the four groups (Adu/SUS/SUS+Adu/sham) based on matching performance on day 5 (final day) of the APA test. A dose of 5 mg/kg Adu was given for each treatment, except for the last treatment where a mixture of 2.5 mg/kg unlabeled Adu and 2.5 mg/kg Alexa Fluor 647-labeled Adu was administered. The mice were initially treated once a week for four weeks, after which they were tested in the APA. From 15-22 months of age the mice were subsequently treated five times, then sacrificed three days following the last treatment, resulting in a total of nine treatments (FIG. 1A).

Aducanumab Analog when Delivered by SUS Improves Spatial Memory Performance

In the current study, we compared the effect of delivering the murine chimeric IgG2a Aducanumab analog, Adu, with a SUS treatment, using plaque burden and behavior as the major read-outs. We also assessed a combination treatment (SUS+Adu). Additional comparisons were made by including sham-treated mice, as well as untreated wild-type littermate controls.

We first tested 13 month-old APP23 mice and their wild-type littermates in the APA test of hippocampus-dependent spatial learning in which the animals must use visual cues to learn to avoid a shock zone located in a rotating arena (FIG. 1B). Spatial learning could have been assessed in the Morris water maze; however, this test is stressful to mice, and aged mice are poor swimmers. To determine the effect of each treatment protocol on spatial memory function, an APA test consisting of 5 training days with a single 10 min training session each day was performed following habituation to the arena in a one 5 min session the day before the first training day. A two-way ANOVA based on the number of shocks that were received revealed a significant effect of day of testing, indicating that learning had occurred (F_{4,208})=5.728, p=0.0003. There was also a significant effect of genotype, with APP23 mice receiving more shocks than their wild-type littermates (F_{1, 52})=6.278, p=0.0154 (FIG. 1C). There was no significant interaction. Similarly, based on the measure of time to first entry of the shock zone, there was a significant effect of day, with mice showing longer latencies to the first entrance as the number of training days increased (F_{4,208})=7.586, p=0.0007. Wild-type mice showed longer latencies to enter the shock zone over the days of testing and there was a significant effect of genotype on time to first entry (F_{1.52})=5.950, p=0.0182 (FIG. 1D). Wild-type and APP23 mice did not differ however on number of entries (FIG. 1E) or maximum time of avoidance (FIG. 1F). APP23 mice performed significantly worse on the measures time to second entry (FIG. 1G) and proportion of time spent in the quadrant opposite to the shocked quadrant (FIG. 1H) The APA performance of the APP23 mice varied significantly so they were assigned to each of the four treatment groups based on matching performance in terms of the number of shocks received on day 5 of the APA to reduce any differences in performance between treatment groups so as to more readily detect any improvement caused by a treatment (FIG. 1I).

Before retesting in the APA, mice were subsequently treated once a week for four weeks. For the retest, the shock zone was shifted by 180 degrees, the cues in the room were changed and the arena rotated in the opposite direction. To perform well in the retest, the mice needed to update their spatial learning in order to learn the new shock zone location (FIG. 1F). A two-way ANOVA with group as a between-subjects factor and day as a repeated measures factor revealed a significant effect of treatment group on number of shocks received (F_{4,47}=8.5, p<0.0001). Follow-up multiple comparisons tests showed that SUS+Adu-treated mice received significantly fewer shocks than sham-treated control mice on days 3 (p=0.0295) and 5 (p=0.0005) (FIG. 1K). Comparing just the Adu-treated to the SUS+Adu-treated mice by two-way ANOVA revealed that the combined treatment of SUS+Aducanumab led to significantly improved performance over Aducanumab only in terms of number of shocks received during the test (F_{1,17}=6.23, p=0.0231). A two-way ANOVA revealed a significant effect of group (F₄,47=6.8, p=0.0002) on time to first entry into the shock zone, with follow-up multiple comparisons test showing that SUS+Adu-treated mice had a longer latency to enter the shock zone on day 5 of the APA task (p=0.024) (FIG. 1L). SUS+Adu-treated mice also performed significantly better than sham-treated mice on the measures number of entries (FIG. 1M) maximum time of avoidance (FIG. 1N), time to second entry (FIG. 1O) and proportion of time spent in the quadrant opposite the shock zone (FIG. 1P). These results demonstrate that APP23 mice exhibit an improvement in spatial memory when treated with a combination of SUS and Adu. Of note, we were interested in determining amyloid pathology at an advanced age; however, it is then not possible to perform a third APA test as mice are of this age are in a poor condition preventing them from physically performing the task.

Comparison of Plaque Reduction in the Cortex for the Different Treatment Groups

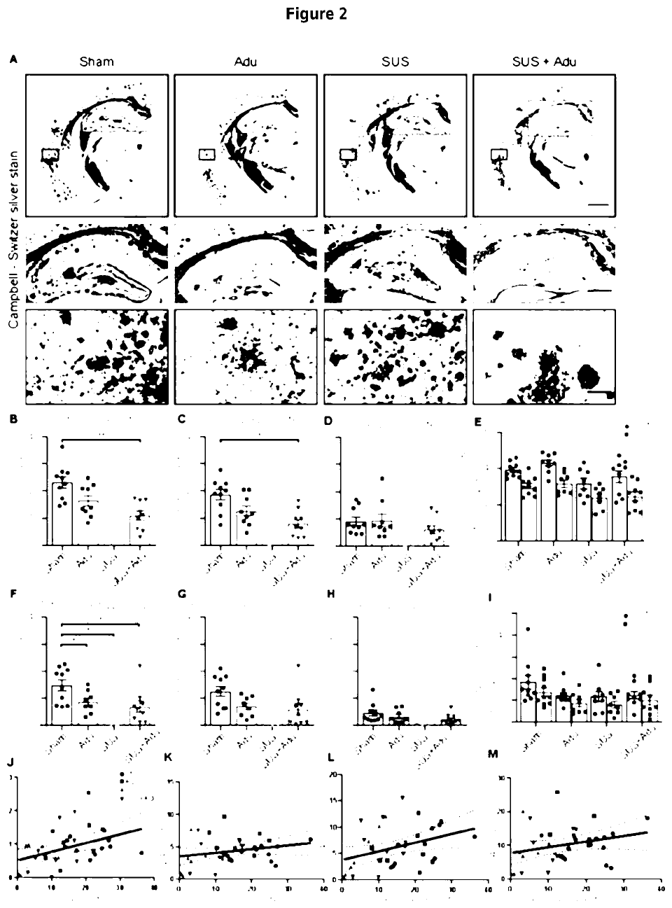

Following APA testing to ascertain the effects of four once-per-week treatments on spatial memory performance, APP23 mice had five treatment sessions between the age of 15 and 22 months in order to determine whether treating them with SUS, Adu alone, or a combination resulted in robust plaque removal, even at older ages when plaque burden is maximal. The mice were sacrificed at 22 months of age, three days after the last treatment and one hemisphere was processed for histology to identify plaques. We performed Campbell-Switzer staining, which can differentiate diffuse and compact species of amyloid plaques in the brain and is not confounded by the binding of Adu to Aβ. This revealed a reduction in the total amount of plaque area stained when comparing the treatment groups to sham controls (FIG. 2A). We calculated the percentage area occupied by plaque for two regions of interest, the cortex and the hippocampus, in 15-20 sections per mouse, assessing plaque burden in a one-in-eight series of sections throughout the rostral-caudal extent of the brain. Analysis of cortical plaque burden in the different groups revealed an effect of treatment (F₃,31=3.78, p=0.02). A follow-up Holm-Sidak test found that combined SUS+Adu treatment resulted in a statistically significant 52% plaque reduction in the cortex of SUS+Adu-treated APP23 mice compared to sham (p=0.0066). At 22 months of age, these mice have a severe plaque burden, with diffuse and compact plaques occupying 23% of the cortex in the sham-treated mice, compared to 16% of the cortex in mice administered Adu, 17% in mice administered SUS only and 11% in mice which received a combination SUS+Adu treatment (FIG. 2B). As the Campbell-Switzer silver differentiates diffuse plaques stained black with a cotton wool appearance, and compact plaques stained amber (FIG. 2A), we also performed an analysis of these plaques separately using a color deconvolution method in ImageJ. The results of this analysis revealed that the reduction in total plaque area was largely driven by a reduction in the total area of black plaque which occupied 18.60% of the cortical area in sham-treated mice compared to 7.93% in SUS+Adu-treated mice (p=0.0119) (FIG. 2C). In contrast, we found no significant difference between the treatment groups based on the area, number, or size of amber plaques (FIG. 2D,E,I). A one-tailed unpaired t-test revealed that the combination of SUS+Adu led to significantly lower plaque burden in the cortex than Aducanumab alone (p=0.029).

Aducanumab Analog, SUS and the Combination Therapy all Effectively Reduce Amyloid Plaques in the Hippocampus of APP23 Mice

In APP23 mice, plaque formation is initiated in the cortex and then proceeds to the hippocampus. When we analyzed the total plaque burden in the hippocampus, we found a significant effect of treatment (F_{3,31}=, p=0.03, one-way ANOVA). All three treatments (Adu, SUS and Adu+SUS) led to a significant reduction in total plaque area in the hippocampus compared to that in sham-treated APP23 mice (FIG. 2F). The sham-treated mice had a hippocampal plaque burden of 14.84% vs 8.68% (p=0.0432) for Adu-treated mice, 8.04% for SUS-treated mice (p=0.043) and 6.92% for SUS+Adu-treated mice (p=0.022). However, unlike the effects seen in the cortex, the reduction in total plaque in the hippocampus was not disproportionately driven by a reduction in black plaque (F_{3,30}=2.43, p=0.08) (FIG. 2G) compared to amber plaque reduction (F_{3,30}=1.80 p=0.17) (FIG. 2H) as it was only when total plaque burden was analyzed that statistically significant reductions in plaques were found. These results show that the effect of SUS on reducing plaque burden in the hippocampus is comparable to that of Aducanumab. Delivering both Aducanumab and SUS together did not significantly improve the plaque clearing ability in the hippocampus of the treatments by themselves.

Effects of Treatment on Amyloid-β Species

We performed ELISA measurements of amyloid-β40 and amyloid-β42 species from the lysate from one cortex, fractionating proteins into a DEA fraction containing soluble proteins and a guanidine fraction containing insoluble proteins. Levels of A342 and A340 in the DEA soluble fraction were significantly correlated to plaque burden as measured by histology Campbell-Switzer silver staining of the other hemisphere (R²=0.17, p=0.014 and R²=0.13, p=0.030 respectively) (FIG. 2J,L). Levels of Aβ42 and Aβ40 in the guanidine fraction containing insoluble Aβ were found not to correlate significantly with plaque burden as measured by histology potentially because most of the material stained by Campbell-Switzer silver staining is soluble in DEA (FIG. 2K,M).

Aducanumab Analog does not Affect Cerebral Amyloid Angiopathy in APP23 Mice

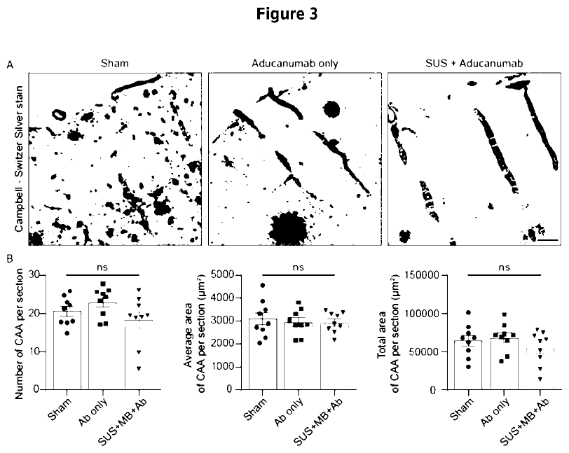

We investigated whether there was any effect of treatment with SUS or Adu or the combination on cerebral amyloid angiopathy (CAA). APP23 mice exhibit amyloid deposition on the vasculature with advanced age. We found that there was no effect of Adu, or a combination of both on the number of blood vessels that were Campbell-Switzer positive and mice in all groups had significant deposition of amyloid on blood vessels (FIG. 3A). An average of 20 vessels were Campbell Switzer positive per section, with an average of 0.75% of the total area of the cortex taken up by amyloid-laden blood vessels and this did not differ between the groups (FIG. 3B).

SUS Markedly Increases the Amount of the Aducanumab Analog in the Brain

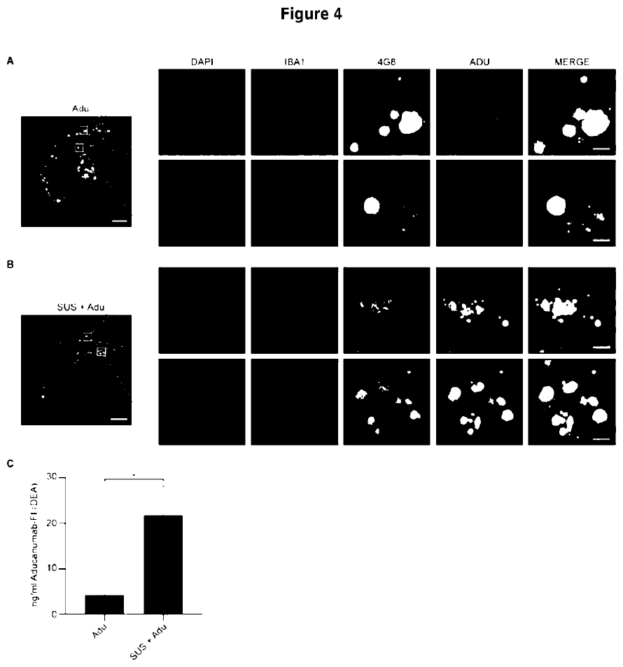

We also sought to determine the extent to which SUS was able to increase the amount of Adu in the brain. We therefore labeled Adu with Alexa Fluor 647 and injected 2.5 mg/kg fluorescently labeled Adu and 2.5 mg/kg unlabeled Adu at the last treatment session. In mice treated with Adu alone, fluorescently labeled Adu was faintly detectable by fluorescence microscopy and mainly confined to the outside edge of plaques (FIG. 4A). In contrast, in SUS+Adu mice, fluorescently labeled Adu decorated the entirety of plaques and was easily detectable (FIG. 4B). We also analyzed a subset of 5 mice per group to determine the area of the cortex that was positive for fluorescent Adu, revealing that 0.36% of the cortex in Adu treated mice was positive compared to 1.59% of the brain in SUS+Adu mice (p=0.0096, t-test). We also detected fluorescently labeled Adu in mice injected with Adu and SUS+Adu in the cortical lysate and found that levels were 4.32 ng/ml on average in the Adu group compared to 21.77 ng/ml in the SUS+Adu mice (p=0.0175, t-test) (FIG. 4C). In summary, the way in which Adu binds to plaques looks very different when combined with SUS compared to Adu alone, such that the entire plaque including the core is totally covered with Adu for SUS+Adu, but with Adu alone the core is not bound by Adu.

Here, the inventors used a multi-arm study, in which we compared the effects of SUS, an Aducanumab analog, Adu, delivered peripherally, and Adu delivered to the brain using SUS in APP23 mice with plaque pathology, using a sham treatment and wild-type mice as controls. It was found that in the treatment paradigm (nine treatments from age 13 to 22 months of age), Adu delivered across the BBB with SUS markedly reduced the amyloid plaque burden in the cortex of 22 month-old APP23 mice compared to the effects of either the antibody or SUS alone. For the first time the inventors report data on the effect of an Aducanumab analog in a spatial memory task in plaque-bearing AD model mice. The inventors performed the APA test of spatial memory and learning in 13 month-old APP23 mice to obtain a baseline, and then divided the mice into treatment groups based on their performance on the test which would allow a greater power to detect improvements caused by the treatment due to reduced variability between the groups. The APP23 mice were treated four times once per week and then repeated the APA test where mice had to learn new spatial cues to avoid the shock zone. This experimental design allowed detection of an improvement in mice treated with the combination of SUS and Aducanumab compared to sham-treated mice, and to detect improved performance in mice treated with combination compared to mice treated with Aducanumab without SUS.

The inventors were also interested in the effect of treatment on amyloid plaque burden, specifically at an advanced age in mice where plaque burden is more similar to an early AD patient, so the mice were aged until 22 months of age. To conserve antibody, the inventors performed five treatments over this period. Interestingly, plaque reduction in the hippocampus could be achieved with any of the three treatments, possibly reflecting the lower degree and later appearance of pathology in this brain area. Spatial memory was improved in 14 month-old APP23 mice following four weekly treatments with a combination of Adu and SUS. The inventors observed a five-fold increased levels of Adu in this study when administered in combination with SUS, which was measured three days after the treatment. The total increased uptake would likely be higher if measured at earlier time points post-injection, as IgG is cleared from the CNS or taken up by microglia as time progresses.

Delivering higher amounts of an anti-Aβ antibody into the brain could increase the efficacy of immunotherapy. The inventors results show that administering a much lower cumulative dose of an Aducanumab analog than previously described is ineffective at clearing plaques in the cortex of APP23 mice when treatment is commenced at 13 months of age; however, plaques in the hippocampus which develop at a more advanced age were reduced. In contrast to peripheral injections alone, delivery of the Aducanumab analog using SUS led to a reduction in both cortical and hippocampal plaques, concomitant with increased brain levels of the antibody.

Although recent findings were disappointing for aducanumab regarding the effects on cognition if amyloid reduction were to be a goal in patients with a high amyloid burden as potentially detected by blood tests, CSF or amyloid PET but no cognitive impairment then approaches that can remove amyloid faster with less injected antibody could be attractive.

Delivering an anti-Abeta antibody to the brain using ultrasound could be an approach to perform prevention trials as it would maximise target engagement of the antibody in the brain, lower the dose and cost required to perform the study and likely minimise the occurrence of adverse events due to interaction of antibody in the periphery.

It will be understood that the invention disclosed and defined in this specification extends to all alternative combinations of two or more of the individual features mentioned or evident from the text or drawings. All of these different combinations constitute various alternative aspects of the invention.

1. A method of delivering an antigen binding site that binds to or specifically binds to an amyloid beta (β) protein in a subject comprising:
administering to the subject an antigen binding site that binds to or specifically binds to an amyloid beta protein, and
administering acoustic energy to a region of the brain of the subject;
wherein the application of acoustic energy acts as a means to permit or facilitate the antigen binding site to pass through the blood-brain barrier (BBB) of the subject, thereby delivering the antigen binding that binds to or specifically binds to an amyloid beta protein.

2. A method according to claim 1, wherein the amyloid beta protein is in a form selected from the group consisting of soluble Aβ, Aβ oligomer or present in amyloid plaques.

3. A method according to claim 2, wherein the amyloid beta protein is a plaque.

4. A method according to any one of claims 1 to 3, wherein the amyloid beta protein is selected from amyloid beta, amyloid fragments, amyloid precursor protein, and amyloid precursor protein fragments.

5. A method according to any one of claims 1 to 4, wherein the acoustic energy is ultrasound.

6. A method according to claim 5, wherein the ultrasound is scanning ultrasound (SUS) or non-scanning ultrasound.

7. A method according to claim 6, wherein the SUS or non-scanning ultrasound is administered with microbubbles to disrupt the blood-brain barrier.

8. A method according to claim 7, wherein the administration of microbubbles may be before, after or during the administration of SUS or non-scanning ultrasound.

9. A method of improving cognitive function in a subject, the method comprising, consisting essentially of or consisting of the steps of:
administering to the subject an antigen binding site that binds to or specifically binds to an amyloid beta protein;
identifying a region of the brain of the subject to which acoustic energy is to be applied; and
applying a clinically safe level of acoustic energy to the region, thereby saturating or substantially saturating the region with acoustic energy;
thereby improving cognitive function in the subject.

10. A method according to claim 9, wherein the subject may have impaired cognitive function.

11. A method according to claim 9 or 10, wherein the method further comprises the step of identifying the subject as having impaired cognitive function.

12. A method of improving cognitive function in a subject with a condition associated with a pathological form of an amyloid beta protein, the method comprising, consisting essentially of or consisting of the steps of:
administering to a subject an antigen binding site that binds to or specifically binds to an amyloid beta protein;
identifying a region of the brain of the subject to which acoustic energy is to be applied; and
applying a clinically safe level of acoustic energy to the region, thereby saturating or substantially saturating the region with acoustic energy;
thereby improving cognitive function in the subject.

13. A method according to any one of claims 9 to 12, wherein the condition or disease for treatment is one associated with or caused by a pathological form of an amyloid beta protein.

14. A method of improving memory, motor skills and/or executive functions in a subject with impaired memory function, the method including the steps of:
administering to a subject an antigen binding site that binds to or specifically binds to an amyloid beta protein;
identifying a region of the brain of the subject to which acoustic energy is to be applied; and
applying a clinically safe level of acoustic energy to the region, thereby saturating or substantially saturating the region with acoustic energy;
thereby improving memory, motor skills and/or executive functions in the subject.

15. A method of improving memory, motor skills, executive functions and/or cognitive function in a subject with impaired memory and/or cognitive function, the method including the steps of:
providing a subject with impaired memory, motor skills, executive functions, and/or cognitive function;
administering to the subject an antigen binding site that binds to or specifically binds to an amyloid beta protein;
identifying a region of the brain of the subject to which acoustic energy is to be applied; and
applying a clinically safe level of acoustic energy to the region, thereby saturating or substantially saturating the region with acoustic energy;
thereby improving memory, motor skills, executive functions and/or cognitive function in the subject.

16. A method according to any one of claims 9 to 15, wherein identifying a region of the brain as described herein includes determining a volume of the brain on the basis of symptoms displayed by the subject, typically clinically observable or biochemically detectable symptoms, or determining a volume of the brain on the basis of a known association with an amyloid beta protein, preferably in pathogenic form, in particular those associated with protein oligomers, aggregates or deposits, or determining a volume of the brain including a volume surrounding an site having an amyloid beta protein in a pathogenic form, such as oligomers, an aggregate or deposit.

17. A method according to any one of claims 9 to 16, wherein the region of the brain is the entire brain, hemisphere, forebrain or a region of the brain of the subject known to be associated with a condition involving the presence of proteins adopting pathogenic structures in an extracellular region.

18. A method according to claim 17, wherein the region may be any one or more of the following cerebrum, cerebral hemisphere, telencephalon, forebrain, cortex, frontal lobe, prefrontal cortex, precentral gyrus, primary motor cortex, premotor cortex, temporal lobe, auditory cortex, inferior temporal cortex, superior temporal gyrus, fusiform gyrus, parahippocampal gyrus, entorhinal cortex, parietal lobe, somatosensory cortex, postcentral gyrus, occipital lobe, visual cortex, insular cortex, cingulate cortex, subcortical, hippocampus, dentate gyrus, cornu ammonis, amygdala, basal ganglia, striatum, caudate, putamen, nucleus accumbens, olfactory tubercle, globus pallidus, subthalamic nuclei, piriform cortex, olfactory bulb, fornix, mammillary bodies, basal forebrain, nucleus basalis Meynert, diencephalon, thalamus, hypothalamus, midbrain, tectum, tegmentum, substantia nigra, hindbrain, myelencephalon, medulla oblongata, metencephalon, pons, cerebellum, spinal cord, brain stem and cranial nerves, preferably, the region is selected from the group consisting of cerebrum, cerebral hemisphere, telencephalon, forebrain, cortex, frontal lobe, prefrontal cortex, precentral gyrus, temporal lobe, auditory cortex, inferior temporal cortex, superior temporal gyrus, fusiform gyrus, parahippocampal gyrus, entorhinal cortex, insular cortex, cingulate cortex, subcortical, hippocampus, dentate gyrus, cornu ammonis, amygdala, piriform cortex, olfactory bulb, fornix, mammillary bodies, basal forebrain and nucleus basalis of Meynert.

19. A method according to any one of claims 1 to 18, wherein the antigen binding site that binds to amyloid-β has an affinity that is not statistically different to the antibody BIIB037 (aducanumab).

20. A method according to any one of claims 1 to 19, wherein the antigen binding site binds the same epitope on amyloid-β as BIIB037 (aducanumab).

21. A method according to any one of claims 1 to 20, wherein the antigen binding site competes with BIIB037 (aducanumab) for binding to amyloid-β.

22. A method according to any one of claims 1 to 21, wherein the antigen binding site comprises, consists essentially of or consists of an antigen binding domain of an antibody, wherein the antigen binding domain binds to or specifically binds to amyloid-beta, wherein the antigen binding domain comprises:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set in SEQ ID NO: 2 and a CDR3 comprising a sequence at least about 80%, at least 85%, at least 90%, at least 92%, at least 95%, at least 97%, at least 99% identical to a sequence set forth in SEQ ID NO: 3;
(ii) a VH comprising a sequence at least about 95% or 96% or 97% or 98% or 99% identical to a sequence set forth in SEQ ID NO: 15;
(iii) a VH comprising a CDR1 comprising a sequence set forth in SEQ ID NO: 1, a CDR2 comprising a sequence set forth between in SEQ ID NO: 2 and a CDR3 comprising a sequence set forth in SEQ ID NO: 3; or
(iv) a VH comprising a sequence set forth in SEQ ID NO: 15;
and,
(v) a VL comprising a CDR1 comprising a sequence at least about 80%, at least 85%, at least 90%, at least 92%, at least 95%, at least 97%, at least 99% identical to a sequence set forth in SEQ ID NO: 4, a CDR2 comprising a sequence at least about 80%, at least 85%, at least 90%, at least 92%, at least 95%, at least 97%, at least 99% identical to a sequence set forth in SEQ ID NO: 5 and a CDR3 comprising a sequence at least about 80%, at least 85%, at least 90%, at least 92%, at least 95%, at least 97%, at least 99% identical to a sequence set forth in SEQ ID NO: 6;
(vi) a VL comprising a sequence at least about 95% identical to a sequence set forth in SEQ ID NO: 16;
(vii) a VL comprising a CDR1 comprising a sequence set SEQ ID NO: 4, a CDR2 comprising a sequence set forth in SEQ ID NO: 5 and a CDR3 comprising a sequence set forth in SEQ ID NO: 6; or
(viii) a VL comprising a sequence set forth in SEQ ID NO: 16.

23. An apparatus configured to perform any one or more of the methods according to claims 1 to 22.

24. Use of an antigen binding site as defined in any one or claims 1 to 22, in the preparation of a medicament for treating, inhibiting, delaying or reducing the progression of a disease or condition associated with amyloid beta protein in a subject in need thereof who has received or who is receiving an application of acoustic energy, preferably scanning ultrasound (SUS).

25. An antigen binding site as defined in any one or claims 1 to 22, for use in treating, inhibiting, delaying or reducing the progression of a disease or condition associated with amyloid beta protein in a subject in need thereof who has received or who is receiving an application of acoustic energy, preferably scanning ultrasound (SUS).
